SEQUENCE LISTING (43 sequences)

SEQ ID NO: 1 - protein, 20 aa, Homo sapiens
  REGION 1..20: note = Description of sequence: human IL-2 signal sequence
MYRMQLLSCIALSLALVTNS

SEQ ID NO: 2 - protein, 20 aa, Mus musculus
  REGION 1..20: note = Description of sequence: mouse IL-2 signal sequence
MYSMQLASCVTLTLVLLVNS

SEQ ID NO: 3 - protein, 20 aa, Homo sapiens
  REGION 1..20: note = Description of sequence: human kappa leader sequence
METPAQLLFLLLLWLPDTTG

SEQ ID NO: 4 - protein, 20 aa, Mus musculus
  REGION 1..20: note = Description of sequence: mouse kappa leader sequence
METDTLLLWVLLLWVPGSTG

SEQ ID NO: 5 - protein, 21 aa, Homo sapiens
  REGION 1..21: note = Description of sequence: human CD8 leader sequence
MALPVTALLLPLALLLHAARP

SEQ ID NO: 6 - protein, 18 aa, Homo sapiens
  REGION 1..18: note = Description of sequence: truncated human CD8 signal peptide MALPVTALLLPLALLLHA
MALPVTALLLPLALLLHA

SEQ ID NO: 7 - protein, 16 aa, Homo sapiens
  REGION 1..16: note = Description of sequence: human albumin signal sequence
MKWVTFISLLFSSAYS

SEQ ID NO: 8 - protein, 30 aa, Homo sapiens
  REGION 1..30: note = Description of sequence: human prolactin signal sequence
MDSKGSSQKGSRLLLLLVVSNLLLCQGVVS

SEQ ID NO: 9 - protein, 281 aa, Homo sapiens
  REGION 1..281: note = Description of sequence: UniProt Reference No.: P48023
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 10 - protein, 258 aa, synthetic construct
  REGION 1..258: note = Description of sequence: FasLdel1
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKEPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 11 - DNA, 774 bp, synthetic construct
  misc_feature 1..774: note = Description of sequence: FasLdel1
  source 1..774: mol_type = other DNA
atgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagcccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctc

SEQ ID NO: 12 - protein, 277 aa, synthetic construct
  REGION 1..277: note = Description of sequence: FasLdel2
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 13 - DNA, 831 bp, synthetic construct
  misc_feature 1..831: note = Description of sequence: FasLdel2
  source 1..831: mol_type = other DNA
atgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctgaagaagagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttgggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctc

SEQ ID NO: 14 - protein, 279 aa, Homo sapiens
  REGION 1..279: note = Description of sequence: UniProt Reference No.: P41047-1
MQQPMNYPCPQIFWVDSSATSSWAPPGSVFPCPSCGPRGPDQRRPPPPPPPVSPLPPPSQPLPLPPLTPLKKKDHNTNLWLPVVFFMVLVALVGMGLGMYQLFHLQKELAELREFTNQSLKVSSFEKQIANPSTPSEKKEPRSVAHLTGNPHSRSIPLEWEDTYGTALISGVKYKKGGLVINETGLYFVYSKVYFRGQSCNNQPLNHKVYMRNSKYPEDLVLMEEKRLNYCTTGQIWAHSSYLGAVFNLTSADHLYVNISQLSLINFEESKTFFGLYKL

SEQ ID NO: 15 - RNA, 20 nt, synthetic construct
  misc_feature 1..20: note = Description of sequence: gRNA Knockout sequence
  source 1..20: mol_type = other RNA
ggagttgatgtcagtcactt

SEQ ID NO: 16 - protein, 220 aa, Homo sapiens
  REGION 1..220: note = Description of sequence: NCBI Reference No: NP_006130
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 17 - protein, 164 aa, Homo sapiens
  REGION 1..164: note = Description of sequence: NCBI Reference No: NP_932170
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 18 - protein, 112 aa, Homo sapiens
  REGION 1..112: note = Description of sequence: Component of CD3-zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 22 aa, Homo sapiens
  REGION 1..22: note = Description of sequence: ITAM1
QNQLYNELNLGRREEYDVLDKR

SEQ ID NO: 20 - DNA, 66 bp, Homo sapiens
  misc_feature 1..66: note = Description of sequence: Native ITAM1
  source 1..66: mol_type = genomic DNA
cagaaccagctctataacgagctcaatctaggacgaagagaggagtacgatgttttggacaagaga

SEQ ID NO: 21 - protein, 22 aa, Homo sapiens
  REGION 1..22: note = Description of sequence: ITAM1 variant
QNQLFNELNLGRREEFDVLDKR

SEQ ID NO: 22 - DNA, 66 bp, Homo sapiens
  misc_feature 1..66: note = Description of sequence: ITAM1 variant
  source 1..66: mol_type = genomic DNA
cagaaccagctctttaacgagctcaatctaggacgaagagaggagttcgatgttttggacaagaga

SEQ ID NO: 23 - protein, 23 aa, Homo sapiens
  REGION 1..23: note = Description of sequence: native ITAM2
QEGLYNELQKDKMAEAYSEIGMK

SEQ ID NO: 24 - DNA, 69 bp, Homo sapiens
  misc_feature 1..69: note = Description of sequence: native ITAM2
  source 1..69: mol_type = genomic DNA
caggaaggcctgtacaatgaactgcagaaagataagatggcggaggcctacagtgagattgggatgaaa

SEQ ID NO: 25 - protein, 23 aa, Homo sapiens
  REGION 1..23: note = Description of sequence: ITAM2 variant
QEGLFNELQKDKMAEAFSEIGMK

SEQ ID NO: 26 - DNA, 69 bp, Homo sapiens
  misc_feature 1..69: note = Description of sequence: ITAM2 variant
  source 1..69: mol_type = genomic DNA
caggaaggcctgttcaatgaactgcagaaagataagatggcggaggccttcagtgagattgggatgaaa

SEQ ID NO: 27 - protein, 22 aa, Homo sapiens
  REGION 1..22: note = Description of sequence: native ITAM3
HDGLYQGLSTATKDTYDALHMQ

SEQ ID NO: 28 - DNA, 66 bp, Homo sapiens
  misc_feature 1..66: note = Description of sequence: native ITAM3
  source 1..66: mol_type = genomic DNA
cacgatggcctttaccagggtctcagtacagccaccaaggacacctacgacgcccttcacatgcag

SEQ ID NO: 29 - protein, 22 aa, Homo sapiens
  REGION 1..22: note = Description of sequence: ITAM3 variant
HDGLFQGLSTATKDTFDALHMQ

SEQ ID NO: 30 - DNA, 66 bp, Homo sapiens
  misc_feature 1..66: note = Description of sequence: ITAM3 variant
  source 1..66: mol_type = genomic DNA
cacgatggccttttccaggggctcagtacagccaccaaggacaccttcgacgcccttcacatgcag

SEQ ID NO: 31 - protein, 112 aa, synthetic construct
  REGION 1..112: note = Description of sequence: Component of CAR 1XX
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 32 - DNA, 336 bp, synthetic construct
  misc_feature 1..336: note = Description of sequence: Component of CAR 1XX
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 33 - DNA, 123 bp, Homo sapiens
  misc_feature 1..123: note = Description of sequence: NCBI Reference No: NP_006130
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 34 - protein, 218 aa, Mus musculus
  REGION 1..218: note = Description of sequence: NCBI Reference No: NP_031668.3
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 35 - protein, 255 aa, Homo sapiens
  REGION 1..255: note = Description of sequence: NCBI Ref. No.: NP_001552
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 36 - protein, 250 aa, synthetic construct
  REGION 1..250: note = Description of sequence: FasLPro
MQQPFNYPYPQIYWVDSSASSPWAPPGTVLPCPTSVPRRPGQRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 37 - DNA, 750 bp, synthetic construct
  misc_feature 1..750: note = Description of sequence: FasLPro
  source 1..750: mol_type = other DNA
atgcagcagcccttcaattacccatatccccagatctactgggtggacagcagtgccagctctccctgggcccctccaggcacagttcttccctgtccaacctctgtgcccagaaggcctggtcaaagagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctc

SEQ ID NO: 38 - protein, 281 aa, synthetic construct
  REGION 1..281: note = Description of sequence: FasLKKR
MQQPFNYPYPQIYWVDSSASSPWAPPGTVLPCPTSVPRRPGQRRPPPPPPPPPLPPPPPPPPLPPLPLPPLEEEGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 39 - DNA, 843 bp, synthetic construct
  misc_feature 1..843: note = Description of sequence: FasLKKR
  source 1..843: mol_type = other DNA
atgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctggaagaagaagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagaagcaaataggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctc

SEQ ID NO: 40 - protein, 278 aa, synthetic construct
  REGION 1..278: note = Description of sequence: FasLKKRdel2
MQQPFNYPYPQIYWVDSSASSPWAPPGTVLPCPTSVPRRPGQRRPPPPPPPPPLPPPPPPPPLPPLPLPPLEEEGNHSTGLCLLVMFFMVLVALVGLGLGMFQLFHLQKELAELRESTSQMHTASSLEGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 41 - DNA, 834 bp, synthetic construct
  misc_feature 1..834: note = Description of sequence: FasLKKRdel2
  source 1..834: mol_type = other DNA
atgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctggaagaagaagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttggagggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctc

SEQ ID NO: 42 - protein, 277 aa, synthetic construct
  REGION 1..277: note = Description of sequence: FasLdel2KKR
MQQPFNYPYPQIYWVDSSASSPWAPPGTVLPCPTSVPRRPGQRRPPPPPPPPPLPPPPPPPPLPPLPLPPLEEEGNHSTGLCLLVMFFMVLVALVGLGLGMFQLFHLQKELAELRESTSQMHTASSLGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 43 - DNA, 831 bp, synthetic construct
  misc_feature 1..831: note = Description of sequence: FasLdel2KKR
  source 1..831: mol_type = other DNA
atgcagcagcccttcaattacccatatccccagatctactgggtggacagcagtgccagctctccctgggcccctccaggcacagttcttccctgtccaacctctgtgcccagaaggcctggtcaaaggaggccaccaccaccaccgccaccgccaccactaccacctccgccgccgccgccaccactgcctccactaccgctgccacccctggaagaagaagggaaccacagcacaggcctgtgtctccttgtgatgtttttcatggttctggttgccttggtaggattgggcctggggatgtttcagctcttccacctacagaaggagctggcagaactccgagagtctaccagccagatgcacacagcatcatctttgggccaccccagtccaccccctgaaaaaaaggagctgaggaaagtggcccatttaacaggcaagtccaactcaaggtccatgcctctggaatgggaagacacctatggaattgtcctgctttctggagtgaagtataagaagggtggccttgtgatcaatgaaactgggctgtactttgtatattccaaagtatacttccggggtcaatcttgcaacaacctgcccctgagccacaaggtctacatgaggaactctaagtatccccaggatctggtgatgatggaggggaagatgatgagctactgcactactgggcagatgtgggcccgcagcagctacctgggggcagtgttcaatcttaccagtgctgatcatttatatgtcaacgtatctgagctctctctggtcaattttgaggaatctcagacgtttttcggcttatataagctc

(19) United States
(12) Patent Application Publication
(10) Pub. No.: US 2024/0350547 A1
(43) Pub. Date: Oct. 24, 2024

(54) Cells Expressing FAS Ligand Polypeptides and FAS Knockout and Uses Thereof

(71) Applicants: Memorial Sloan-Kettering Cancer Center, New York, NY (US); Sloan-Kettering Institute for Cancer Research, New York, NY (US); Memorial Hospital for Cancer and Allied Diseases, New York, NY (US)

(72) Inventors: Michel Sadelain, New York, NY (US); Karlo Perica, New York, NY (US)

(73) Assignees: Memorial Sloan-Kettering Cancer Center, New York, NY (US); Sloan-Kettering Institute for Cancer Research, New York, NY (US); Memorial Hospital for Cancer and Allied Diseases, New York, NY (US)

(21) Appl. No.: 18/749,852

(22) Filed: Jun. 21, 2024

Related U.S. Application Data
(63) Continuation of application No. PCT/US2022/053750, filed on Dec. 22, 2022.
(60) Provisional application No. 63/292,834, filed on Dec. 22, 2021.

(51) Int. Cl. A61K 35/17; A61K 39/00; A61P 35/00; C07K 14/705; C07K 14/725

(52) U.S. Cl. CPC A61K 35/17; A61K 39/4611; A61K 39/4631; A61K 39/464438; A61P 35/00; C07K 14/7051; C07K 14/70575

(57) ABSTRACT

The presently disclosed subject matter provides cells, compositions and methods for enhancing immune responses toward tumor. It relates to cells comprising: an antigen-recognizing receptor (e.g., a chimeric antigen receptor, a TCR, or a TCR like fusion molecule), a Fas ligand polypeptide (FasL), and a gene disruption of a Fas locus. The gene disruption of the Fas locus can improve the activity and/or efficiency of the cells. The presently disclosed cells, compositions, and methods can be used in allogeneic settings.

### CROSS-REFERENCE TO RELATED APPLICATIONS

This application is a Continuation Application of International Patent Application No. PCT/US2022/053750, filed Dec. 22, 2022, which claims priority to U.S. Provisional Patent Application No. 63/292,834, filed on Dec. 22, 2021, the content of each of which is incorporated by reference in its entirety, and to each of which priority is claimed.

### SEQUENCE LISTING

A Sequence Listing conforming to the rules of WIPO Standard ST.26 is hereby incorporated by reference. Said Sequence Listing has been filed as an electronic document via EFS-Web in ASCII format encoded as XML. The electronic document, created on Jun. 20, 2024, is entitled “0727341635.xml”, and is 65,536 bytes in size.

### 1. INTRODUCTION

The presently disclosed subject matter provides cells, compositions and methods for enhancing immune responses toward tumors and immune cells. It relates to cells comprising an antigen-recognizing receptor (e.g., a chimeric antigen receptor or a TCR like fusion molecule), a Fas Ligand (FasL) polypeptide and a gene disruption of a Fas locus. The gene disruption of the Fas locus can improve the activity and/or efficiency of the cells.

### 2. BACKGROUND OF THE INVENTION

Cell-based immunotherapy is a therapy with curative potential for the treatment of cancer. T cells and other immune cells may be modified to target tumor antigens through the introduction of genetic material coding for an antigen-recognizing receptor, e.g., a Chimeric antigen receptor or a TCR like fusion molecule. Patient-engineered CAR T cells have demonstrated remarkable efficacy against a range of liquid and solid malignancies. However, treatment failure and relapses occur in a large fraction of patients. Therefore, there remain needs of improved immunotherapy.

### 3. SUMMARY OF THE INVENTION

The presently disclosed subject matter provides cells comprising a FasL polypeptide and a gene disruption of a Fas locus. In certain embodiments, the cells further comprise an antigen-recognizing receptor (e.g., a CAR, a TCR, or a TCR like fusion molecule). The presently disclosed cells can be used for cell lysis of target cells expressing Fas, and for treating diseases or disorders, e.g., tumors.

In certain embodiments, the presently disclosed subject matter provides an immunoresponsive cell comprising an exogenous Fas ligand polypeptide (FasL) and a gene disruption of a Fas locus. In certain embodiments, the immunoresponsive cell further comprises an antigen-recognizing receptor that targets an antigen.

In certain embodiments, the FasL polypeptide is capable of binding to Fas. In certain embodiments, the FasL polypeptide is membrane bound. In certain embodiments, the FasL polypeptide comprises an amino acid sequence that is at least about 80% identical to the amino acid sequence set forth in SEQ ID NO: 10. In certain embodiments, the FasL polypeptide comprises the amino acid sequence set forth in SEQ ID NO: 10. In certain embodiments, the FasL polypeptide comprises an amino acid sequence that is at least about 80% identical to the amino acid sequence set forth in SEQ ID NO: 12. In certain embodiments, the FasL polypeptide comprises the amino acid sequence set forth in SEQ ID NO: 12. In certain embodiments, the FasL polypeptide comprises or consists of an amino acid sequence that is at least about 80% identical to the amino acid sequence set forth in SEQ ID NO: 36. In certain embodiments, the FasL polypeptide comprises or consists of the amino acid sequence set forth in SEQ ID NO: 36. In certain embodiments, the FasL polypeptide comprises or consists of an amino acid sequence that is at least about 80% identical to the amino acid sequence set forth in SEQ ID NO: 38. In certain embodiments, the FasL polypeptide comprises or consists of the amino acid sequence set forth in SEQ ID NO: 38. In certain embodiments, the FasL polypeptide comprises or consists of an amino acid sequence that is at least about 80% identical to the amino acid sequence set forth in SEQ ID NO: 40. In certain embodiments, the FasL polypeptide comprises or consists of the amino acid sequence set forth in SEQ ID NO: 40. In certain embodiments, the FasL polypeptide comprises or consists of an amino acid sequence that is at least about 80% identical to the amino acid sequence set forth in SEQ ID NO: 42. In certain embodiments, the FasL polypeptide comprises or consists of the amino acid sequence set forth in SEQ ID NO: 42. In certain embodiments, the FasL polypeptide comprises a truncated intracellular domain. In certain embodiments, the FasL polypeptide does not comprise an intracellular domain. In certain embodiments, the FasL polypeptide comprises or consists of an amino acid sequence of amino acids 81 to 281 of SEQ ID NO: 9. In certain embodiments, the FasL polypeptide comprises or consists of an amino acid sequence of amino acids 81 to 277 of SEQ ID NO: 12. In certain embodiments, the FasL polypeptide is expressed from a vector.

In certain embodiments, the gene disruption comprises a mutation, a substitution, a deletion, an insertion, or a combination thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof. In certain embodiments, the gene disruption of the Fas locus results in a non-functional Fas protein. In certain embodiments, the gene disruption of the Fas locus results in knockout of the Fas gene expression. In certain embodiments, the gene disruption of the Fas locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the antigen-recognizing receptor is a recombinant T cell receptor (TCR), a chimeric antigen receptor (CAR), or a TCR like fusion molecule. In certain embodiments, the antigen-recognizing receptor is a CAR. In certain embodiments, the antigen-recognizing receptor is encoded by a polynucleotide inserted into a first locus within the genome. In certain embodiments, the first locus is selected from the group consisting of a TRAC locus, a TRBC locus, a TRDC locus, a TRGC locus, and a Fas locus. In certain embodiments, the first locus is a Fas locus. In certain embodiments, the first locus is a TRAC locus.

In certain embodiments, the FasL polypeptide is encoded by a polynucleotide inserted into a second locus within the genome. In certain embodiments, the second locus is selected from the group consisting of a TRAC locus, a TRBC locus, a TRDC locus, a TRGC locus, and a Fas locus. In certain embodiments, the second locus is a Fas locus. In certain embodiments, the second locus is a TRAC locus.

In certain embodiments, the antigen-recognizing receptor and the FasL polypeptide arc encoded by a polynucleotide inserted into a first locus within the genome. In certain embodiments, the first locus is selected from the group consisting of a TRAC locus, a TRBC locus, a TRDC locus, a TRGC locus, and a Fas locus. In certain embodiments, the first locus is a TRAC locus.

In certain embodiments, the antigen is a tumor antigen or a pathogen antigen. In certain embodiments, the antigen is a tumor antigen. In certain embodiments, the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinasc3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, and ERBB.

In certain embodiments, the cell further comprises a gene disruption of a TRAC locus, a TRBC locus, a TRDC locus, and a TRGC locus, or a combination thereof. In certain embodiments, the gene disruption comprises a substitution, a deletion, an insertion, or a combination thereof. In certain embodiments, the gene disruption results in a non-functional protein or in knockout of the gene expression. In certain embodiments, the gene disruption is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the cell further comprises a gene disruption of a B2M locus. In certain embodiments, the gene disruption of the B2M locus results in a non-functional beta 2-microglobulin. In certain embodiments, the gene disruption of the B2M locus results in knockout of the B2M gene expression. In certain embodiments, the gene disruption of the B2M locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the cell further comprises a gene disruption of a CIITA locus. In certain embodiments, the gene disruption of the CIITA locus results in a non-functional MHC class II transactivator. In certain embodiments, the gene disruption of the CIITA locus results in knockout of the CIITA gene expression. In certain embodiments, the gene disruption of the CIITA locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the Fas locus is capable of enhancing at least one activity of the cell comprising the antigen-recognizing receptor. In certain embodiments, the at least one activity comprises cytotoxicity, cell proliferation, cell persistence, or a combination thereof.

In certain embodiments, the immunoresponsive cell is a cell of the lymphoid lineage or a cell of the myeloid lineage. In certain embodiments, the cell is selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof. In certain embodiments, the cell is a T cell. Natural Killer (NK) cell Natural Killer (NK) cell. In certain embodiments, the cell is autologous. In certain embodiments, the cell is allogeneic.

The presently disclosed subject matter further provides a composition comprising the immunoresponsive cells disclosed herein. In certain embodiments, the composition is a pharmaceutical composition further comprising a pharmaceutically acceptable excipient.

The presently disclosed subject matter also provides a method for producing the cell disclosed herein. In certain embodiments, the method comprises: generating a gene disruption of a Fas locus in a cell. In certain embodiments, the method further comprises introducing into the cell a polynucleotide encoding a FasL polypeptide. In certain embodiments, the cell comprises an antigen-recognizing receptor.

In certain embodiments, the gene disruption comprises a mutation, a substitution, a deletion, an insertion, or a combination thereof. In certain embodiments, the mutation comprises s a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof. In certain embodiments, the gene disruption of the Fas locus results in a non-functional Fas protein. In certain embodiments, the gene disruption of the Fas locus results in knockout of the Fas gene expression. In certain embodiments, generating the gene disruption of the Fas locus comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the FasL polypeptide comprises an amino acid sequence that is at least about 80% identical to the amino acid sequence set forth in SEQ ID NO: 10. In certain embodiments, the FasL polypeptide comprises the amino acid sequence set forth in SEQ ID NO: 10. In certain embodiments, the FasL polypeptide comprises an amino acid sequence that is at least about 80% identical to the amino acid sequence set forth in SEQ ID NO: 12. In certain embodiments, the FasL polypeptide comprises the amino acid sequence set forth in SEQ ID NO: 12. In certain embodiments, the FasL polypeptide comprises or consists of an amino acid sequence that is at least about 80% identical to the amino acid sequence set forth in SEQ ID NO: 36. In certain embodiments, the FasL polypeptide comprises or consists of the amino acid sequence set forth in SEQ ID NO: 36. In certain embodiments, the FasL polypeptide comprises or consists of an amino acid sequence that is at least about 80% identical to the amino acid sequence set forth in SEQ ID NO: 38. In certain embodiments, the FasL polypeptide comprises or consists of the amino acid sequence set forth in SEQ ID NO: 38. In certain embodiments, the FasL polypeptide comprises or consists of an amino acid sequence that is at least about 80% identical to the amino acid sequence set forth in SEQ ID NO: 40. In certain embodiments, the FasL polypeptide comprises or consists of the amino acid sequence set forth in SEQ ID NO: 40. In certain embodiments, the FasL polypeptide comprises or consists of an amino acid sequence that is at least about 80% identical to the amino acid sequence set forth in SEQ ID NO: 42. In certain embodiments, the FasL polypeptide comprises or consists of the amino acid sequence set forth in SEQ ID NO: 42. In certain embodiments, the FasL polypeptide comprises a truncated intracellular domain. In certain embodiments, the FasL polypeptide does not comprise an intracellular domain. In certain embodiments, the FasL polypeptide comprises or consists of an amino acid sequence of amino acids 81 to 281 of SEQ ID NO: 9. In certain embodiments, the FasL polypeptide comprises or consists of an amino acid sequence of amino acids 81 to 277 of SEQ ID NO: 12.

In certain embodiments, the method further comprises generating a gene disruption of a TRAC locus in the cell. In certain embodiments, the gene disruption of the TRAC locus results in a non-functional T cell receptor (TCR). In certain embodiments, the gene disruption of the TRAC locus results in knockout of the TCR gene expression. In certain embodiments, generating the gene disruption of the TRAC locus to the cell comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the method further comprises generating a gene disruption of a B2M locus in the cell. In certain embodiments, the gene disruption of the B2M locus results in a non-functional beta 2-microglobulin. In certain embodiments, the gene disruption of the B2M locus results in knockout of the B2M gene expression. In certain embodiments, generating the gene disruption of the B2M locus to the cell comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the method further comprises generating a gene disruption of a CIITA locus in the cell. In certain embodiments, the gene disruption of the CIITA locus results in a non-functional MHC class II transactivator. In certain embodiments, the gene disruption of the CIITA locus results in knockout of the CIITA gene expression. In certain embodiments, generating the gene disruption of the CIITA locus to the cell comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

The presently disclosed subject matter also provides a cell produced by the methods disclosed herein.

Moreover, the presently disclosed subject matter provides a nucleic acid composition comprising a first polynucleotide encoding a Fas ligand polypeptide, and a second polynucleotide encoding a nuclease. In certain embodiments, the FasL polypeptide comprises or consists of an amino acid sequence that is at least about 80% identical to the amino acid sequence set forth in SEQ ID NO: 10, SEQ ID NO: 12, SEQ ID NO: 36, SEQ ID NO: 38, SEQ ID NO: 40, or SEQ ID NO: 42. In certain embodiments, the FasL polypeptide comprises or consists of an amino acid sequence set forth in SEQ ID NO: 10, SEQ ID NO: 12, SEQ ID NO: 36, SEQ ID NO: 38, SEQ ID NO: 40, or SEQ ID NO: 42. In certain embodiments, the FasL polypeptide comprises a truncated intracellular domain. In certain embodiments, the FasL polypeptide does not comprise an intracellular domain. In certain embodiments, the FasL polypeptide comprises or consists of an amino acid sequence of amino acids 81 to 281 of SEQ ID NO: 9. In certain embodiments, the FasL polypeptide comprises or consists of an amino acid sequence of amino acids 81 to 277 of SEQ ID NO: 12.

In certain embodiments, the nuclease is selected from the group consisting of a Zinc finger nuclease, a meganuclease, a Transcription activator-like effector nuclease (TALEN), and a Cas nuclease. In certain embodiments, the nucleic acid composition further comprises a third polynucleotide comprising a gRNA. In certain embodiments, the gRNA comprises or consists of the nucleotide sequence set forth in SEQ ID NO: 15.

In certain embodiments, the nucleic acid composition further comprises a fourth polynucleotide encoding an antigen-recognizing receptor that binds to an antigen. In certain embodiments, the antigen-recognizing receptor is a TCR, a CAR, or a TCR like fusion molecule. In certain embodiments, the antigen-recognizing receptor is a CAR.

The presently disclosed subject matter also provides lipid nanoparticles comprising the nucleic acid composition discloses herein. Also provided are cells comprising nucleic acid compositions or lipid nanoparticles disclosed herein. In addition, the presently disclosed subject matter provides methods of lysing a target cell expressing Fas. In certain embodiments, the method comprises comprising contacting the target cell with the cells or compositions disclosed herein. In certain embodiments, the target cell comprises a tumor cell. In certain embodiments, the target cell comprises an immune cell. In certain embodiments, the immune cell comprises a T cell, a Natural Killer (NK) cell, or a combination thereof.

Furthermore, the presently disclosed subject matter provides methods of reducing tumor burden, preventing and/or treating a tumor, and treating a disease or a disorder in a subject. In certain embodiments, the method comprises administering to the subject an effective amount of the cells or compositions disclosed herein. In certain embodiments, the method reduces the number of tumor cells, reduces tumor size, and/or eradicates the tumor in the subject. In certain embodiments, the disease or disorder is selected from tumors, pathogen infections, autoimmune diseases, and infectious diseases. In certain embodiments, the disease or disorder is a tumor. In certain embodiments, the cell or composition reduces tumor burden, induces tumor cell death, reduces the number of tumor cells, reduces tumor size, and/or eradicates the tumor in the subject. In certain embodiments, the tumor is a solid tumor. In certain embodiments, the tumor is a hematological tumor. In certain embodiments, the tumor is a cancer. In certain embodiments, the disease or disorder is a pathogen infection or an infectious disease. In certain embodiments, the disease or disorder is an autoimmune disease. In certain embodiments, the subject is human.

### 4. BRIEF DESCRIPTION OF THE FIGURES

The following Detailed Description, given by way of example, but not intended to limit the presently disclosed subject matter to specific embodiments described, may be

FIGS. 1A-1G illustrate that adoptive cell therapies (e.g., CAR T cells) for allogeneic infusion can be generated but they are sensitive to immune rejection. FIG. 1A shows a schematic to study immune rejection of allogeneic CAR T cells. FIG. 1B shows expression of CD3ε in T cells expressing a chimeric antigen receptor (CAR) and edited with a Cas9 and a TRAC-directed guide RNA. FIG. 1C shows tumor growth in mice infused with CAR T cells and autologous or allogeneic human peripheral blood mononuclear cells (PBMCs). FIG. 1D shows total amount of CAR T cells found in bone marrow of mice infused with allogeneic or autologous PBMCs. FIG. 1E shows a schematic of an in vitro assay for monitoring immune rejection of CARs. FIG. 1F shows CAR T cell survival in the presence of freshly isolated autologous or allogeneic PBMCs. FIG. 1G shows CAR T cell survival in presence of MLR-stimulated autologous or allogeneic PBMCs.

FIGS. 2A-2E show multiple CRISPR-Cas9 edited CAR T cells can be generated and can evade immunity. FIG. 2A shows a schematic of allogeneic host-versus-graft immunity against traditional CAR T cells. FIG. 2B shows expression of TCR/CD3, MHC Class I, and/or MHC Class II in CAR T cells edited with multiple CRISPR-Cas9. FIG. 2C shows the survival of edited CAR T cells co-cultured in vitro with CD8 T cells. FIG. 2C shows the survival of edited CAR T cells co-cultured in vitro with NK cells. TRAC: edited CAR T cells lacking TCR expression; TRAC+B2m: edited CAR T cells lacking TCR and beta-2-microglobulin expression; *p<0.05 by t-test (paired). FIG. 2E shows tumor growth in mice infused with edited CAR T cells and autologous and allogeneic PBMCs.

FIGS. 3A-3D show methods for designing adoptive cell therapies (e.g., CAR T cells) that are resistant to host-versus-graft immunity. FIG. 3A shows strategies to overcome host-versus-graft immunity. FIG. 3B shows a schematic of gene editing of adoptive cell therapies (e.g., CAR T cells). FIG. 3C shows retroviral vector strategies. FIG. 3D shows AAV vector strategies.

FIGS. 4A-4D show that Fas knockout protected CAR T cells from allogeneic killing. FIG. 4A shows TCR and Fas expression in CAR T cells edited with CRISPR-Cas9 sgRNA targeting either TRAC locus alone (TRAC KO) or TRAC and Fas (TRAC+Fas KO). FIG. 4B shows a screening of Cas9 sgRNAs. Exemplary SEQ ID NO: 15 is represented. FIG. 4C shows FasL-mediated apoptosis of edited CAR T cells lacking Fas (Fas KO) or DR5 (DR5 KO). FIG. 4D shows survival curves of CAR T cells edited with CRISPR-Cas9 sgRNA targeting either TRAC locus alone (TRAC KO) or TRAC and Fas (TRAC+Fas KO) when incubated with autologous PBMCs. FIG. 4D shows survival curves of CAR T cells edited with CRISPR-Cas9 sgRNA targeting either TRAC locus alone (TRAC KO) or TRAC and Fas (TRAC+Fas KO) when incubated with allogeneic PBMCs.

FIGS. 5A-5E show that active tolerance can be promoted through overexpression of Fas Ligand (FasL), FasL variants, and other protective proteins. FIG. 5A shows a schematic of FasL metalloproteinase domain and proline rich domain. Del1 refers to a FasL polypeptide comprising or consisting of the amino acid sequence set forth in SEQ ID NO: 10. Del2 refers to a FasL polypeptide comprising or consisting of the amino acid sequence set forth in SEQ ID NO: 12. DelPro refers to a FasL polypeptide including a deletion of amino acid 43 to 73. KKR refers to a FasL polypeptide including a deletion of amino acid 71 to 73. FIG. 5B shows bicistronic vectors containing FasL variants and EGFR. FIG. 5C shows expression levels of FasL detected by fluorescence staining four days after transduction with retroviral vectors encoding FasL. FIG. 5D shows survival of edited CAR T cells in presence of MLR-stimulated allogeneic PBMCs for 24 hours. FIG. 5E shows increased expression of 1928z CAR and FasL after transduction with AAV bicistronic vectors containing 1928z CAR and/or FasL. FIG. 5E shows increased expression of 1928z CAR and FasL polypeptides after transduction with AAV bicistronic vectors containing 1928z CAR and FasL, 19282 CAR and FasLdel2, or 1928z CAR and FasLPro.

### 5. DETAILED DESCRIPTION OF THE INVENTION

The presently disclosed subject matter provides cells comprising i) a Fas ligand (FasL) polypeptide, and ii) a gene disruption of a Fas locus. In certain embodiments, the cells further comprise an antigen-recognizing receptor (e.g., a TCR or a CAR). The presently disclosed subject matter also provides methods of using such cells for lysing target cells expressing Fas, and for treating diseases or disorders, e.g., tumors, infectious diseases, autoimmune diseases, etc. The presently disclosed subject matter is based, at least in part, on the discovery that cells comprising a FasL polypeptide and a gene disruption of a Fas locus can overcome host-versus-graft by promoting Fas-FasL-mediated lysis of target immune cells expressing Fas (e.g., endogenous T cells or NK cells targeting the presently disclosed cells) and avoiding fratricide killing by means of the gene disruption of the Fas locus within the cell.

Non-limiting embodiments of the presently disclosed subject matter are described by the present specification and Examples.

For purposes of clarity of disclosure and not by way of limitation, the detailed description is divided into the following subsections:
5.1. Definitions;
5.2. Fas ligand polypeptides;
5.3. Gene disruption of a Fas locus;
5.4. Antigen-Recognizing Receptors;
5.5. Cells;
5.6. Nucleic Acid Compositions and Vectors;
5.7. Formulations and Administration; and
5.8. Methods of Treatment

### 5.1. Definitions

Unless defined herein, all technical and scientific terms used in this detailed description have the meaning commonly understood by a person skilled in the art of immune oncology as reflected, for example, in general definitions of many of the terms used in the presently disclosed subject matter included in one or more of the following: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof, including cells that initiate, activate, and/or regulate (increase or decrease) an immune response.

By “activates an immunoresponsive cell” is meant induction of signal transduction or changes in protein expression in the cell resulting in initiation of an immune response. For example, when CD3 chains cluster in response to ligand binding and immunoreceptor tyrosine-based inhibition motifs (ITAMs), a signal transduction cascade is produced. In certain embodiments, binding of a TCR or a CAR to an antigen leads to a formation of an immunological synapse that includes clustering of many molecules near the bound receptor (e.g. CD4 or CD8, CD3γ/δ/ε/ζ, etc.). This clustering of membrane bound signaling molecules allows ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or is concomitantly mediated through receptors including, but not limited to, CD28, CD137 (4-1BB), OX40, CD40 and ICOS. Receiving multiple stimulatory signals can be important to mount a robust and long-term T-cell mediated immune response, but T cells receiving multiple stimulatory signals can quickly become inhibited and unresponsive to antigen, a state commonly referred to as “exhaustion”. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of recognizing a target antigen. In certain embodiments, the antigen-recognizing receptor is capable of activating an immune or immunoresponsive cell (e.g., a T cell) upon its binding to the target antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., Nucl Med (1983); 24:316-325). As used herein, include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, C_{H}1, C_{H}2 and C_{H}3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987), or IMGT numbering system (Lefranc, The Immunologist (1999); 7:132-136; Lefranc et al., Dev. Comp. Immunol. (2003); 27:55-77). The CDRs can also be numbered according to the IMGT numbering system, e.g., the IMGT numbering system accessible at http://www.imgt.org/IMGT_vquest/input. Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated according to the Kabat numbering system.

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. “Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains).

Despite removal of the constant regions and the introduction of a linker, scFv proteins retain the specificity of the original immunoglobulin. Single chain Fv polypeptide antibodies can be expressed from a nucleic acid including V_{H}- and V_{L} encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27 (6): 455-51; Peter et al., J Cachexia Sarcopenia Muscle 2012 Aug. 12; Shich et al., J Imunol2009 183 (4): 2277-85; Giomarelli et al., Thromb Haemost 2007 97 (6): 955-63; Fife eta., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chern 2003 25278 (38): 36740-7; Xie et al., Nat Biotech 1997 15 (8): 768-71; Ledbetter et al., Crit Rev Immunol1997 17 (5-6): 427-55; Ho et al., BioChim Biophys Acta 2003 1638 (3): 257-66).

As used herein, “F (ab)” refers to a fragment of an antibody structure that binds to an antigen but is monovalent and does not have a Fc portion, for example, an antibody digested by the enzyme papain yields two F (ab) fragments and an Fc fragment (e.g., a heavy (H) chain constant region; Fc region that does not bind to an antigen).

As used herein, “F(ab′)₂” refers to an antibody fragment generated by pepsin digestion of whole IgG antibodies, wherein this fragment has two antigen binding (ab′) (bivalent) regions, wherein each (ab′) region comprises two separate amino acid chains, a part of a H chain and a light (L) chain linked by an S—S bond for binding an antigen and where the remaining H chain portions are linked together. A “F(ab′)₂” fragment can be split into two individual Fab′ fragments.

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule (e.g., a synthetic receptor) comprising an extracellular antigen-binding domain fused to an intracellular signaling domain that is capable of activating or stimulating an immunoresponsive cell. In certain embodiments, the CAR further comprises a transmembrane domain. In certain embodiments, the extracellular antigen-binding domain of a CAR comprises an scFv. The scFv can be derived from fusing the variable heavy and light regions of an antibody. In certain embodiments, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” includes any nucleic acid molecule that encodes a polypeptide of interest (e.g., a Fas ligand polypeptide, or an antigen-recognizing receptor) or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule.

By “substantially identical” or “substantially homologous” is meant an amino acid sequence or a nucleic acid molecule exhibiting at least about 50% homologous or identical to a reference amino acid sequence (for example, any one of the amino acid sequences described herein) or a reference nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the reference amino acid or the reference nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

In certain embodiments, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. The term “constitutive expression” or “constitutively expressed” as used herein refers to expression or expressed under all physiological conditions.

As used herein, the term “vector” refers to any genetic element, such as a plasmid, phage, transposon, cosmid, chromosome, virus, virion, etc., which is capable of replication when associated with the proper control elements and which can transfer gene sequences into cells. Thus, the term includes cloning and expression vehicles, as well as viral vectors and plasmid vectors.

By “disease” is meant any condition, disease or disorder that damages or interferes with the normal function of a cell, tissue, or organ, e.g., neoplasm, and pathogen infection of cell.

An “effective amount” (or, “therapeutically effective amount”) is an amount sufficient to affect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder. By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, a human IL-2 signal sequence (e.g. MYRMQLLSCIALSLALVTNS [SEQ ID NO: 1]), a mouse IL-2 signal sequence (e.g., MYSMQLASCVTLTLVLLVNS [SEQ ID NO: 2]), a human kappa leader sequence (e.g., METPAQLLFLLLLWLPDTTG [SEQ ID NO: 3]), a mouse kappa leader sequence (e.g., METDTLLLWVLLLWVPGSTG [SEQ ID NO: 4]); a human CD8 leader sequence (e.g., MALPVTALLLPLALLLHAARP [SEQ ID NO: 5]); a truncated human CD8 signal peptide (e.g., MALPVTALLLPLALLLHA [SEQ ID NO: 6]); a human albumin signal sequence (e.g., MKWVTFISLLESSAYS [SEQ ID NO: 7]); and a human prolactin signal sequence (e.g., MDSKGSSQKGSRLLLLLVVSNLLLCQGVVS [SEQ ID NO: 8]).

In certain embodiments, the CAR comprises a CD8 signal peptide at the N-terminus, e.g., the signal peptide is connected to the extracellular antigen-binding domain of the CAR. In certain embodiments, the CD8 signal peptide comprises or consists of the amino acid sequence set forth in SEQ ID NO: 6.

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigenic substance produced in tumor cells. Tumor antigens can trigger an immune response in the host. As used herein, the term “tumor antigen” includes tumor-specific antigens (TSAs) and tumor-associated antigens (TAAs). In certain embodiments, TSAs comprise antigens that are uniquely or differentially expressed on a tumor cell as compared to a normal cell, e.g., present only on tumor cells and not on normal cells. In certain embodiments, a tumor antigen includes any polypeptide expressed by a tumor that is capable of activating or inducing an immune response via an antigen-recognizing receptor or capable of suppressing an immune response via receptor-ligand binding. TAAs are antigens that are present on some tumor cells and also some normal cells.

The terms “comprises”, “comprising”, and are intended to have the broad meaning ascribed to them in U.S. Patent Law and can mean “includes”, “including” and the like.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system. In certain embodiments, the subject is a human.

Other aspects of the presently disclosed subject matter are described in the following disclosure and are within the ambit of the presently disclosed subject matter.

### 5.2. Fas Ligand Polypeptides

Fas ligand (FasL) is a member of the TNF-ligand superfamily and is a type II transmembrane protein. FasL binds to Fas, which induces apoptosis. Fas is involved in the regulation of cytotoxic T cell-mediated apoptosis, natural killer cell-mediated apoptosis and in T-cell development. FasL initiates fratricidal/suicidal activation-induced cell death (AICD) in antigen-activated T cells contributing to the termination of immune response. The interaction of FasL with its receptor Fas allows the formation of a cell death-inducing signaling complex with other components, e.g., Fas-associated protein with death domain (FADD), which can induce programmed cell death, also known as apoptosis. The cell death-inducing property of FasL is associated with its extracellular domain, which can be cleaved off by metalloprotease activity to produce soluble FasL.

The presently disclosed immunoresponsive cells comprise an exogenous FasL polypeptide. In certain embodiments, the presently disclosed immunoresponsive cells overexpress a FasL polypeptide. In certain embodiments, the presently disclosed FasL polypeptide is constitutively expressed on the surface of immunoresponsive cells (e.g., T cells or NK cells). In certain embodiments, the expression of the presently disclosed FasL polypeptide on the immunoresponsive cells is initiated upon antigen-specific activation of the cells.

FasL comprises an extracellular domain, a transmembrane domain, and an intracellular domain.

In certain embodiments, the FasL polypeptide is a human FasL polypeptide. In certain embodiments, the human FasL comprises or consists of the amino acid sequence of UniProt Reference No.: P48023-1 (SEQ ID NO: 9). SEQ ID NO: 9 is provided below. In certain embodiments, the intracellular domain or cytoplasmic domain of human FasL comprises or consists of amino acids 1 to 80 of SEQ ID NO: 9. In certain embodiments, the transmembrane domain of human FasL comprises or consists of amino acids 81 to 102 of SEQ ID NO: 9. In certain embodiments, the extracellular domain of human FasL comprises or consists of amino acids 103 to 281 of SEQ ID NO: 9.

[SEQ ID NO: 9]
MQQPFNYPYPQIYWVDSSASSPWAPPGTVLPCPTSVPRRPGQRRPPPPPP
PPPLPPPPPPPPLPPLPLPPLKKRGNHSTGLCLLVMFFMVLVALVGLGLG
MFQLFHLQKELAELRESTSQMHTASSLEKQIGHPSPPPEKKELRKVAHLT
GKSNSRSMPLEWEDTYGIVLLSGVKYKKGGLVINETGLYFVYSKVYFRGQ
SCNNLPLSHKVYMRNSKYPQDLVMMEGKMMSYCTTGQMWARSSYLGAVFN
LTSADHLYVNVSELSLVNFEESQTFFGLYKL

In certain embodiments, the FasL polypeptide comprises an intracellular domain and a transmembrane domain of human FasL. In certain embodiments, the human FasL polypeptide comprises or consists of the amino acid sequence set forth in SEQ ID NO: 9 or a fragment thereof. In certain embodiments, the human FasL polypeptide comprises or consists of a fragment of the amino acid sequence set forth in SEQ ID NO: 9. In certain embodiments, the human FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 9 or a fragment thereof. In certain embodiments, the FasL polypeptide comprises or consists of an amino acid sequence that is a consecutive portion of SEQ ID NO: 9, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 281 amino acids in length. In certain embodiments, the FasL polypeptide comprises or consists of an amino acid sequence of amino acids 1 to 281, 1 to 80, 81 to 102, 1 to 102, 1 to 110, 1 to 127, 81 to 102, 103 to 281, 132 to 281, 134 to 281, or 135 to 281 of SEQ ID NO: 9. In certain embodiments, the FasL polypeptide comprises or consists of an amino acid sequence of amino acids 1 to 80 of SEQ ID NO: 9. In certain embodiments, the FasL polypeptide comprises or consists of an amino acid sequence of amino acids 81 to 102 of SEQ ID NO: 9. In certain embodiments, the FasL polypeptide comprises or consists of amino acids 1 to 110 of SEQ ID NO: 9. In certain embodiments, the FasL polypeptide comprises or consists of amino acids 1 to 127 of SEQ ID NO: 9. In certain embodiments, the FasL polypeptide comprises or consists of amino acids 132 to 281 of SEQ ID NO: 9. In certain embodiments, the FasL polypeptide comprises or consists of amino acids 134 to 281 of SEQ ID NO: 9. In certain embodiments, the FasL polypeptide comprises or consists of amino acids 135 to 281 of SEQ ID NO: 9.

In certain embodiments, the FasL polypeptide comprises or consists of the amino acid sequence set forth in SEQ ID NO: 10. In certain embodiments, the FasL polypeptide comprising or consisting of the amino acid sequence set forth in SEQ ID NO: 10 or a fragment thereof is also designated as “FasLdel1”. SEQ ID NO: 10 is provided below.

[SEQ ID NO: 10]
MQQPFNYPYPQIYWVDSSASSPWAPPGTVLPCPTSVPRRPGQRRPPPPPP
PPPLPPPPPPPPLPPLPLPPLKKRGNHSTGLCLLVMFFMVLVALVGLGLG
MFQLFHLQKEPSPPPEKKELRKVAHLTGKSNSRSMPLEWEDTYGIVLLSG
VKYKKGGLVINETGLYFVYSKVYFRGQSCNNLPLSHKVYMRNSKYPQDLV
MMEGKMMSYCTTGQMWARSSYLGAVFNLTSADHLYVNVSELSLVNFEESQ
TFFGLYKL

In certain embodiments, the FasL polypeptide comprises or consists of the amino acid sequence set forth in SEQ ID NO: 10 or a fragment thereof. In certain embodiments, the FasL polypeptide comprises or consists of a fragment of the amino acid sequence set forth in SEQ ID NO: 10. In certain embodiments, the FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 10 or a fragment thereof. In certain embodiments, the FasL polypeptide comprises or consists of an amino acid sequence that is a consecutive portion of SEQ ID NO: 10, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 258 amino acids in length. In certain embodiments, the FasL polypeptide comprises or consists of an amino acid sequence of amino acids 1 to 258, 1 to 80, 81 to 102, 1 to 102, 103 to 258, 112 to 258, 113 to 259, or 114 to 258 of SEQ ID NO: 10.

An exemplary nucleotide sequence encoding the amino acid sequence of SEQ ID NO: 10 is set forth in SEQ ID NO: 11, which is provided below.

[SEQ ID NO: 11]
atgcagcagcccttcaattacccatatccccagatctactgggtggacag
cagtgccagctctccctgggcccctccaggcacagttcttccctgtccaa
cctctgtgcccagaaggcctggtcaaaggaggccaccaccaccaccgcca
ccgccaccactaccacctccgccgccgccgccaccactgcctccactacc
gctgccacccctgaagaagagagggaaccacagcacaggcctgtgtctcc
ttgtgatgtttttcatggttctggttgccttggtaggattgggcctgggg
atgtttcagctcttccacctacagaaggagcccagtccaccccctgaaaa
aaaggagctgaggaaagtggcccatttaacaggcaagtccaactcaaggt
ccatgcctctggaatgggaagacacctatggaattgtcctgctttctgga
gtgaagtataagaagggtggccttgtgatcaatgaaactgggctgtactt
tgtatattccaaagtatacttccggggtcaatcttgcaacaacctgcccc
tgagccacaaggtctacatgaggaactctaagtatccccaggatctggtg
atgatggaggggaagatgatgagctactgcactactgggcagatgtgggc
ccgcagcagctacctgggggcagtgttcaatcttaccagtgctgatcatt
tatatgtcaacgtatctgagctctctctggtcaattttgaggaatctcag
acgtttttcggcttatataagctc

In certain embodiments, the FasL polypeptide comprises or consists of the amino acid sequence set forth in SEQ ID NO: 12. In certain embodiments, the FasL polypeptide comprising or consisting of the amino acid sequence set forth in SEQ ID NO: 12 or a fragment thereof is also designated as “FasLdel2”. SEQ ID NO: 12 is provided below.

[SEQ ID NO: 12]
MQQPFNYPYPQIYWVDSSASSPWAPPGTVLPCPTSVPRRPGQRRPPPPPP
PPPLPPPPPPPPLPPLPLPPLKKRGNHSTGLCLLVMFFMVLVALVGLGLG
MFQLFHLQKELAELRESTSQMHTASSLGHPSPPPEKKELRKVAHLTGKSN
SRSMPLEWEDTYGIVLLSGVKYKKGGLVINETGLYFVYSKVYFRGQSCNN
LPLSHKVYMRNSKYPQDLVMMEGKMMSYCTTGQMWARSSYLGAVFNLTSA
DHLYVNVSELSLVNFEESQTFFGLYKL

In certain embodiments, the FasL polypeptide comprises or consists of the amino acid sequence set forth in SEQ ID NO: 12 or a fragment thereof. In certain embodiments, the FasL polypeptide comprises or consists of a fragment of the amino acid sequence set forth in SEQ ID NO: 12. In certain embodiments, the FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 12 or a fragment thereof. In certain embodiments, the FasL polypeptide comprises or consists of an amino acid sequence that is a consecutive portion of SEQ ID NO: 12, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 277 amino acids in length. In certain embodiments, the FasL polypeptide comprises or consists of an amino acid sequence of amino acids 1 to 277, 1 to 80, 1 to 127, 103 to 277, or 128 to 277 of SEQ ID NO: 12.

An exemplary nucleotide sequence encoding the amino acid sequence of SEQ ID NO: 12 is set forth in SEQ ID NO: 13, which is provided below.

[SEQ ID NO: 13]
atgcagcagcccttcaattacccatatccccagatctactgggtggacagcagtgccagctctccctgggccc
ctccaggcacagttcttccctgtccaacctctgtgcccagaaggcctggtcaaaggaggccaccaccaccacc
gccaccgccaccactaccacctccgccgccgccgccaccactgcctccactaccgctgccacccctgaagaag
agagggaaccacagcacaggcctgtgtctccttgtgatgtttttcatggttctggttgccttggtaggattgg
gcctggggatgtttcagctcttccacctacagaaggagctggcagaactccgagagtctaccagccagatgca
cacagcatcatctttgggccaccccagtccaccccctgaaaaaaaggagctgaggaaagtggcccatttaaca
ggcaagtccaactcaaggtccatgcctctggaatgggaagacacctatggaattgtcctgctttctggagtga
agtataagaagggtggccttgtgatcaatgaaactgggctgtactttgtatattccaaagtatacttccgggg
tcaatcttgcaacaacctgcccctgagccacaaggtctacatgaggaactctaagtatccccaggatctggtg
atgatggaggggaagatgatgagctactgcactactgggcagatgtgggcccgcagcagctacctgggggcag
tgttcaatcttaccagtgctgatcatttatatgtcaacgtatctgagctctctctggtcaattttgaggaatc
tcagacgtttttcggcttatataagctc

In certain embodiments, the FasL polypeptide comprises or consists of the amino acid sequence set forth in SEQ ID NO: 36. In certain embodiments, the FasL polypeptide comprising or consisting of the amino acid sequence set forth in SEQ ID NO: 36 or a fragment thereof is also designated as “FasLPro”. SEQ ID NO: 36 is provided below.

[SEQ ID NO: 36]
MQQPFNYPYPQIYWVDSSASSPWAPPGTVLPCPTSVPRRPGQRGNHSTGLCLLVMFFMVLVALVGLGLGMFQL
FHLQKELAELRESTSQMHTASSLEKQIGHPSPPPEKKELRKVAHLTGKSNSRSMPLEWEDTYGIVLLSGVKYK
KGGLVINETGLYFVYSKVYFRGQSCNNLPLSHKVYMRNSKYPQDLVMMEGKMMSYCTTGQMWARSSYLGAVEN
LTSADHLYVNVSELSLVNFEESQTFFGLYKL

In certain embodiments, the FasL polypeptide comprises or consists of the amino acid sequence set forth in SEQ ID NO: 36 or a fragment thereof. In certain embodiments, the FasL polypeptide comprises or consists of a fragment of the amino acid sequence set forth in SEQ ID NO: 36. In certain embodiments, the FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 36 or a fragment thereof. In certain embodiments, the FasL polypeptide comprises or consists of an amino acid sequence that is a consecutive portion of SEQ ID NO: 36, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 250 amino acids in length. the FasL polypeptide comprises or consists of an amino acid sequence of amino acids 1 to 250, 1 to 49, 1 to 96, 72 to 250, or 97 to 250 of SEQ ID NO: 36.

An exemplary nucleotide sequence encoding the amino acid sequence of SEQ ID NO: 36 is set forth in SEQ ID NO: 37, which is provided below.

[SEQ ID NO: 37]
atgcagcagcccttcaattacccatatccccagatctactgggtggacagcagtgccagctctccctgggccc
ctccaggcacagttcttccctgtccaacctctgtgcccagaaggcctggtcaaagagggaaccacagcacagg
cctgtgtctccttgtgatgtttttcatggttctggttgccttggtaggattgggcctggggatgtttcagctc
ttccacctacagaaggagctggcagaactccgagagtctaccagccagatgcacacagcatcatctttggaga
agcaaataggccaccccagtccaccccctgaaaaaaaggagctgaggaaagtggcccatttaacaggcaagtc
caactcaaggtccatgcctctggaatgggaagacacctatggaattgtcctgctttctggagtgaagtataag
aagggtggccttgtgatcaatgaaactgggctgtactttgtatattccaaagtatacttccggggtcaatctt
gcaacaacctgcccctgagccacaaggtctacatgaggaactctaagtatccccaggatctggtgatgatgga
ggggaagatgatgagctactgcactactgggcagatgtgggcccgcagcagctacctgggggcagtgttcaat
cttaccagtgctgatcatttatatgtcaacgtatctgagctctctctggtcaattttgaggaatctcagacgt
ttttcggcttatataagctc

In certain embodiments, the FasL polypeptide comprises or consists of the amino acid sequence set forth in SEQ ID NO: 38. In certain embodiments, the FasL polypeptide comprising or consisting of the amino acid sequence set forth in SEQ ID NO: 38 or a fragment thereof is also designated as “FasLKKR”. SEQ ID NO: 38 is provided below.

[SEQ ID NO: 38]
MQQPFNYPYPQIYWVDSSASSPWAPPGTVLPCPTSVPRRPGQRRPPPPPPPPPLPPPPPPPPLPPLPLPPLEE
EGNHSTGLCLLVMFFMVLVALVGLGLGMFQLFHLQKELAELRESTSQMHTASSLEKQIGHPSPPPEKKELRKV
AHLTGKSNSRSMPLEWEDTYGIVLLSGVKYKKGGLVINETGLYFVYSKVYFRGQSCNNLPLSHKVYMRNSKYP
QDLVMMEGKMMSYCTTGQMWARSSYLGAVENLTSADHLYVNVSELSLVNFEESQTFFGLYKL

In certain embodiments, the FasL polypeptide comprises or consists of the amino acid sequence set forth in SEQ ID NO: 38 or a fragment thereof. In certain embodiments, the FasL polypeptide comprises or consists of a fragment of the amino acid sequence set forth in SEQ ID NO: 38. In certain embodiments, the FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 38 or a fragment thereof. In certain embodiments, the FasL polypeptide comprises or consists of an amino acid sequence that is a consecutive portion of SEQ ID NO: 38, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 281 amino acids in length. In certain embodiments, the FasL polypeptide comprises or consists of an amino acid sequence of amino acids 1 to 281, 1 to 80, 1 to 127, 103 to 281, or 128 to 281 of SEQ ID NO: 38.

An exemplary nucleotide sequence encoding the amino acid sequence of SEQ ID NO: 38 is set forth in SEQ ID NO: 39, which is provided below.

[SEQ ID NO: 39]
atgcagcagcccttcaattacccatatccccagatctactgggtggacagcagtgccagctctccctgggccc
ctccaggcacagttcttccctgtccaacctctgtgcccagaaggcctggtcaaaggaggccaccaccaccacc
gccaccgccaccactaccacctccgccgccgccgccaccactgcctccactaccgctgccacccctgGAAGAA
GAAgggaaccacagcacaggcctgtgtctccttgtgatgtttttcatggttctggttgccttggtaggattgg
gcctggggatgtttcagctcttccacctacagaaggagctggcagaactccgagagtctaccagccagatgca
cacagcatcatctttggagaagcaaataggccaccccagtccaccccctgaaaaaaaggagctgaggaaagtg
gcccatttaacaggcaagtccaactcaaggtccatgcctctggaatgggaagacacctatggaattgtcctgc
tttctggagtgaagtataagaagggtggccttgtgatcaatgaaactgggctgtactttgtatattccaaagt
atacttccggggtcaatcttgcaacaacctgcccctgagccacaaggtctacatgaggaactctaagtatccc
caggatctggtgatgatggaggggaagatgatgagctactgcactactgggcagatgtgggcccgcagcagct
acctgggggcagtgttcaatcttaccagtgctgatcatttatatgtcaacgtatctgagctctctctggtcaa
ttttgaggaatctcagacgtttttcggcttatataagctc

In certain embodiments, the FasL polypeptide comprises or consists of the amino acid sequence set forth in SEQ ID NO: 40. In certain embodiments, the FasL polypeptide comprising or consisting of the amino acid sequence set forth in SEQ ID NO: 40 or a fragment thereof is also designated as “FasLKKRdel2”. SEQ ID NO: 40 is provided below.

[SEQ ID NO: 40]
MQQPFNYPYPQIYWVDSSASSPWAPPGTVLPCPTSVPRRPGQRRPPPPPPPPPLPPPPPPPPLPPLPLPPLEE
EGNHSTGLCLLVMFFMVLVALVGLGLGMFQLFHLQKELAELRESTSQMHTASSLEGHPSPPPEKKELRKVAHL
TGKSNSRSMPLEWEDTYGIVLLSGVKYKKGGLVINETGLYFVYSKVYFRGQSCNNLPLSHKVYMRNSKYPQDL
VMMEGKMMSYCTTGQMWARSSYLGAVENLTSADHLYVNVSELSLVNFEESQTFFGLYKL

In certain embodiments, the FasL polypeptide comprises or consists of the amino acid sequence set forth in SEQ ID NO: 40 or a fragment thereof. In certain embodiments, the FasL polypeptide comprises or consists of a fragment of the amino acid sequence set forth in SEQ ID NO: 40. In certain embodiments, the FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 40 or a fragment thereof. In certain embodiments, the FasL polypeptide comprises or consists of an amino acid sequence that is a consecutive portion of SEQ ID NO: 40, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 278 amino acids in length. In certain embodiments, the FasL polypeptide comprises or consists of an amino acid sequence of amino acids 1 to 277, 1 to 80, 1 to 127, 103 to 277, or 128 to 278 of SEQ ID NO: 40.

An exemplary nucleotide sequence encoding the amino acid sequence of SEQ ID NO: 40 is set forth in SEQ ID NO: 41, which is provided below.

[SEQ ID NO: 41]
atgcagcagcccttcaattacccatatccccagatctactgggtggacagc
agtgccagctctccctgggcccctccaggcacagttcttccctgtccaacc
tctgtgcccagaaggcctggtcaaaggaggccaccaccaccaccgccaccg
ccaccactaccacctccgccgccgccgccaccactgcctccactaccgctg
ccacccctgGAAGAAGAAgggaaccacagcacaggcctgtgtctccttgtg
atgtttttcatggttctggttgccttggtaggattgggcctggggatgttt
cagctcttccacctacagaaggagctggcagaactccgagagtctaccagc
cagatgcacacagcatcatctttggagggccaccccagtccaccccctgaa
aaaaaggagctgaggaaagtggcccatttaacaggcaagtccaactcaagg
tccatgcctctggaatgggaagacacctatggaattgtcctgctttctgga
gtgaagtataagaagggtggccttgtgatcaatgaaactgggctgtacttt
gtatattccaaagtatacttccggggtcaatcttgcaacaacctgcccctg
agccacaaggtctacatgaggaactctaagtatccccaggatctggtgatg
atggaggggaagatgatgagctactgcactactgggcagatgtgggcccgc
agcagctacctgggggcagtgttcaatcttaccagtgctgatcatttatat
gtcaacgtatctgagctctctctggtcaattttgaggaatctcagacgttt
ttcggcttatataagctc

In certain embodiments, the FasL polypeptide comprises or consists of the amino acid sequence set forth in SEQ ID NO: 42. In certain embodiments, the FasL polypeptide comprising or consisting of the amino acid sequence set forth in SEQ ID NO: 42 or a fragment thereof is also designated as “FasLdel2KKR”. SEQ ID NO: 42 is provided below.

[SEQ ID NO: 42]
MQQPENYPYPQIYWVDSSASSPWAPPGTVLPCPTSVPRRPGQRRPPPPPP
PPPLPPPPPPPPLPPLPLPPLEEEGNHSTGLCLLVMFFMVLVALVGLGLG
MFQLFHLQKELAELRESTSQMHTASSLGHPSPPPEKKELRKVAHLTGKSN
SRSMPLEWEDTYGIVLLSGVKYKKGGLVINETGLYFVYSKVYFRGQSCNN
LPLSHKVYMRNSKYPQDLVMMEGKMMSYCTTGQMWARSSYLGAVENLTSA
DHLYVNVSELSLVNFEESQTFFGLYKL

In certain embodiments, the FasL polypeptide comprises or consists of the amino acid sequence set forth in SEQ ID NO: 42 or a fragment thereof. In certain embodiments, the FasL polypeptide comprises or consists of a fragment of the amino acid sequence set forth in SEQ ID NO: 42. In certain embodiments, the FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 42 or a fragment thereof. In certain embodiments, the FasL polypeptide comprises or consists of an amino acid sequence that is a consecutive portion of SEQ ID NO: 42, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 277 amino acids in length. In certain embodiments, the FasL polypeptide comprises or consists of an amino acid sequence of amino acids 1 to 277, 1 to 80, 1 to 127, 103 to 277, or 128 to 277 of SEQ ID NO: 42.

An exemplary nucleotide sequence encoding the amino acid sequence of SEQ ID NO: 42 is set forth in SEQ ID NO: 43, which is provided below.

[SEQ ID NO: 41]
atgcagcagcccttcaattacccatatccccagatctactgggtggacag
cagtgccagctctccctgggcccctccaggcacagttcttccctgtccaa
cctctgtgcccagaaggcctggtcaaaggaggccaccaccaccaccgcca
ccgccaccactaccacctccgccgccgccgccaccactgcctccactacc
gctgccacccctgGAAGAAGAAgggaaccacagcacaggcctgtgtctcc
ttgtgatgtttttcatggttctggttgccttggtaggattgggcctgggg
atgtttcagctcttccacctacagaaggagctggcagaactccgagagtc
taccagccagatgcacacagcatcatctttgggccaccccagtccacccc
ctgaaaaaaaggagctgaggaaagtggcccatttaacaggcaagtccaac
tcaaggtccatgcctctggaatgggaagacacctatggaattgtcctgct
ttctggagtgaagtataagaagggtggccttgtgatcaatgaaactgggc
tgtactttgtatattccaaagtatacttccggggtcaatcttgcaacaac
ctgcccctgagccacaaggtctacatgaggaactctaagtatccccagga
tctggtgatgatggaggggaagatgatgagctactgcactactgggcaga
tgtgggcccgcagcagctacctgggggcagtgttcaatcttaccagtgct
gatcatttatatgtcaacgtatctgagctctctctggtcaattttgagga
atctcagacgtttttcggcttatataagctc

In certain embodiments, the FasL polypeptide is a murine FasL polypeptide. In certain embodiments, the murine FasL comprises or consists of the amino acid sequence of UniProt Reference No.: P41047-1 (SEQ ID NO: 14). SEQ ID NO: 14 is provided below. In certain embodiments, the intracellular domain of murine FasL comprises or consists of amino acids 1 to 78 of SEQ ID NO: 14. In certain embodiments, the transmembrane domain of human FasL comprises or consists of amino acids 79 to 100 of SEQ ID NO: 14. In certain embodiments, the extracellular domain of human FasL comprises or consists of amino acids 101 to 279 of SEQ ID NO: 14.

[SEQ ID NO: 14]
MQQPMNYPCPQIFWVDSSATSSWAPPGSVFPCPSCGPRGPDQRRPPPPPP
PVSPLPPPSQPLPLPPLTPLKKKDHNTNLWLPVVFFMVLVALVGMGLGMY
QLFHLQKELAELREFTNQSLKVSSFEKQIANPSTPSEKKEPRSVAHLTGN
PHSRSIPLEWEDTYGTALISGVKYKKGGLVINETGLYFVYSKVYFRGQSC
NNQPLNHKVYMRNSKYPEDLVLMEEKRLNYCTTGQIWAHSSYLGAVENLT
SADHLYVNISQLSLINFEESKTFFGLYKL

In certain embodiments, the FasL polypeptide comprises an extracellular domain of murine FasL.

In certain embodiments, the murine FasL polypeptide comprises or consists of the amino acid sequence set forth in SEQ ID NO: 14 or a fragment thereof. In certain embodiments, the murine FasL polypeptide comprises or consists of a fragment of the amino acid sequence set forth in SEQ ID NO: 14. In certain embodiments, the murine FasL polypeptide comprises or consists of an amino acid sequence that is at least about 80%, at least about 85%, at least about 90%, at least about 95%, at least about 96%, at least about 97%, at least about 98%, at least about 99%, or at least about 100% identical to the amino acid sequence set forth in SEQ ID NO: 14 or a fragment thereof. In certain embodiments, the FasL polypeptide comprises or consists of an amino acid sequence that is a consecutive portion of SEQ ID NO: 14, which is at least about 20, or at least about 30, or at least about 40, or at least about 50, at least about 60, at least about 70, at least about 80, at least about 90, at least about 100, at least about 110, at least about 120, at least about 130, at least about 140, at least about 150, at least about 160, at least about 170, or at least about 180, and up to about 279 amino acids in length. In certain embodiments, a FasL polypeptide comprises or consists of an amino acid sequence of amino acids 1 to 279, 1 to 78, 1 to 100, 79 to 100, 101 to 279, or 128 to 279 of SEQ ID NO: 14. In certain embodiments, the FasL polypeptide comprises or consists of an amino acid sequence of amino acids 101 to 279 of SEQ ID NO: 14.

In certain embodiments, the FasL polypeptide is a membrane FasL (“mFasL”). As used herein, the term “membrane FasL polypeptide” or “mFasL” refers to the membrane-bound form of a FasL polypeptide. Membrane FasL is resistant to cleavage from a protease or metalloproteinase, thereby avoiding toxicity associated with systemic circulating FasL that can be resulted from cleavage by a protease or a metalloproteinase. In certain embodiments, the FasL polypeptide comprises an intracellular domain, a transmembrane domain, and an extracellular domain that comprises a deletion, wherein the deletion leads to the resistance of the FasL polypeptide to cleavage. In certain embodiments, the mFasL polypeptide comprises or consists of the amino acid sequence set forth in SEQ ID NO: 10. In certain embodiments, the mFasL polypeptide comprises or consists of the amino acid sequence set forth in SEQ ID NO: 12. In certain embodiments, the mFasL polypeptide comprises or consists of the amino acid sequence set forth in SEQ ID NO: 36. In certain embodiments, the mFasL polypeptide comprises or consists of the amino acid sequence set forth in SEQ ID NO: 38. In certain embodiments, the mFasL polypeptide comprises or consists of the amino acid sequence set forth in SEQ ID NO: 40. In certain embodiments, the mFasL polypeptide comprises or consists of the amino acid sequence set forth in SEQ ID NO: 42. In certain embodiments, the mFasL polypeptide comprises a truncated intracellular domain. In certain embodiments, the mFasL polypeptide does not comprise an intracellular domain. In certain embodiments, the mFasL polypeptide comprises or consists of an amino acid sequence of amino acids 81 to 281 of SEQ ID NO: 9. In certain embodiments, the mFasL polypeptide comprises or consists of an amino acid sequence of amino acids 81 to 277 of SEQ ID NO: 12.

In certain embodiments, the FasL polypeptide comprises a truncated intracellular domain. In certain embodiments, the truncated intracellular domain is about 10 amino acids in length, about 20 amino acids in length, about 30 amino acids in length, about 40 amino acids in length, about 50 amino acids in length, about 60 amino acids in length, or about 70 amino acids in length. In certain embodiments, the FasL polypeptide comprises or consists of an amino acid sequence of amino acids 70 to 281 of SEQ ID NO: 9. In certain embodiments, the FasL polypeptide comprises or consists of an amino acid sequence of amino acids 50 to 281 of SEQ ID NO: 9. In certain embodiments, the FasL polypeptide comprises or consists of an amino acid sequence of amino acids 30 to 281 of SEQ ID NO: 9. In certain embodiments, the FasL polypeptide comprises or consists of an amino acid sequence of amino acids 70 to 277 of SEQ ID NO: 12. In certain embodiments, the FasL polypeptide comprises or consists of an amino acid sequence of amino acids 50 to 277 of SEQ ID NO: 12. In certain embodiments, the FasL polypeptide comprises or consists of an amino acid sequence of amino acids 30 to 277 of SEQ ID NO: 12.

In certain embodiments, the FasL polypeptide does not comprise an intracellular domain. In certain embodiments, the FasL polypeptide comprises or consists of an amino acid sequence of amino acids 81 to 281 of SEQ ID NO: 9. In certain embodiments, the FasL polypeptide comprises or consists of an amino acid sequence of amino acids 81 to 277 of SEQ ID NO: 12.

### 5.3. Gene Disruption of a Fas Locus

The presently disclosed subject matter provides cells comprising a gene disruption of a Fas locus. The inventors discovered that gene disruption of a Fas locus along with overexpression of a FasL polypeptide disclosed herein (as disclosed in Section 5.2) in T cells comprising an antigen-recognizing receptor (e.g., a chimeric antigen receptor (CAR) or a TCR like fusion molecule) can protect the T cells from fratricide or suicide killing and lead to the killing of endogenous T cells and NK cells. Therefore, a gene disruption of a Fas locus in the cell comprising such antigen-recognizing receptor and such FasL polypeptide can improve at least one activity of the cells, e.g., cytotoxicity, cell proliferation, and/or cell persistence, and can be used in allogeneic settings.

The gene disruption of the Fas locus can result in a non-functional Fas protein or a knockout of the Fas gene expression. In certain embodiments, the gene disruption of the Fas locus results in knockout of the Fas gene expression.

Non-limiting examples of gene disruptions include mutations, substitutions, deletions, insertions, or combinations thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof.

In certain embodiments, the Fas locus is a human Fas locus. The gene disruption of the Fas locus can be generated by any suitable gene editing methods. In certain embodiments, the gene disruption of the Fas locus (e.g., knockout of the Fas locus) is generated using a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adena-associated viral vectors, vaccinia viruses, bovine papilloma viruses, and herpes viruses (e.g., such as Epstein-Barr Virus).

In certain embodiments, the gene disruption of the Fas locus (e.g., knockout of the Fas locus) is generated using a non-viral method. Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to generate the gene disruption of the Fas locus. In certain embodiments, the gene disruption of the Fas locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a CRISPR system is used to generate the gene disruption of the Fas locus.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

In certain embodiments, the Fas locus is disrupted using a gRNA to knockout expression of Fas. In certain embodiments, the gRNA to knockout the expression of Fas comprises or consists of the nucleotide sequence set forth in SEQ ID NO: 15. SEQ ID NO: 15 is provided below:

[SEQ ID NO: 15]
GGAGUUGAUGUCAGUCACUU

In certain embodiments, zinc-finger nucleases are used to generate the gene disruption of the Fas locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to generate the gene disruption of the Fas locus. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the gene disruption of the Fas locus can be a disruption of the coding region of the Fas locus and/or a disruption of the non-coding region of the Fas locus. In certain embodiments, the gene disruption of the Fas locus comprises a disruption of the coding region of the Fas locus. In certain embodiments, the gene disruption of the Fas locus comprises an insertion at the coding region of the Fas locus. Human Fas protein comprises nine (9) exons: exon 1, exon 2, exon 3, exon 4, exon 5, exon 6, exon 7, exon 8, and exon 9. In certain embodiments, the gene disruption of the Fas locus comprises a disruption at one or more of exon 1 through exon 9 of the Fas locus. In certain embodiments, the gene disruption of the Fas locus comprises a disruption at exon 2 of the Fas locus. In certain embodiments, the gene disruption of the Fas locus comprises an insertion at exon 2 of the Fas locus.

In certain embodiments, the gene disruption of the Fas locus is produced prior to the expression of the antigen-recognizing receptor in the cell. In certain embodiments, the gene disruption of the Fas locus is produced prior to the expression of the FasL polypeptide in the cell.

### 5.4. Antigen-Recognizing Receptors

In certain embodiments, the presently disclosed cells further comprise an antigen-recognizing receptor that binds to an antigen. The subject matter of the instant application, e.g., cells comprising a FasL polypeptide, a gene disruption of a Fas locus, and expression of an antigen-recognizing receptor, finds use irrespective of the particular antigen-recognizing receptor. In certain embodiments, the antigen-recognizing receptor is a chimeric antigen receptor (CAR). In certain embodiments, the antigen-recognizing receptor is a T-cell receptor (TCR). In certain embodiments, the antigen-recognizing receptor is a TCR like fusion molecule. The antigen-recognizing receptor can bind to a tumor antigen or a pathogen antigen. In certain embodiments, the antigen-recognizing receptor binds to a tumor antigen. In certain embodiments, the tumor antigen is a tumor-specific antigen or a tumor-associated antigen.

### 5.4.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, and ERBB. In certain embodiments, the tumor antigen is CD19.

In certain embodiments, the antigen-recognizing receptor binds to a pathogen antigen, e.g., for use in treating and/or preventing a pathogen infection. Non-limiting examples of pathogens include viruses, bacteria, fungi, parasites, and protozoans capable of causing disease.

Non-limiting examples of pathogenic viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses), human papilloma virus (i.e. HPV), JC virus, Epstein Bar Virus, Merkel cell polyoma virus.

Non-limiting examples of pathogenic bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Clostridium difficile, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 5.4.2. T-Cell Receptor (TCR)

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains comprising a Variable (V) region and a Constant (C) region. The Constant region is proximal to the cell membrane, followed by a transmembrane region and a short cytoplasmic tail that lacks the ability to transduce a signal. The Variable region binds to the peptide/MHC complex. The variable domain of each pair (alpha/beta or gamma/delta) of TCR polypeptides comprises three complementarity determining regions (CDRs).

In certain embodiments, a TCR can form a receptor complex with three dimeric signaling modules CD3δ/ε, CD3γ/ε and CD3ζ/ζ or ζ/n. When a TCR complex engages with its antigen and MHC (peptide/MHC), the T cell expressing the TCR complex is activated.

In certain embodiments, the TCR is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the recombinant TCR differs from any naturally occurring TCR by at least one amino acid residue. In certain embodiments, the recombinant TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the recombinant TCR is modified from a naturally occurring TCR by at least one amino acid residue. In certain embodiments, the recombinant TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

In certain embodiments, the TCR recognizes a viral antigen. In certain embodiments, the TCR is expressed in a virus-specific T cell. In certain embodiments, the virus-specific T cell is derived from an individual immune to a viral infection, e.g., BK virus, human herpesvirus 6, Epstein-Barr virus (EBV), cytomegalovirus or adenovirus. In certain embodiments, the virus-specific T cell is a T cell disclosed in Leen et al., Blood, Vol. 121, No. 26, 2013; Barker et al., Blood, Vol. 116, No. 23, 2010; Tzannou et al., Journal of Clinical Oncology, Vol. 35, No. 31, 2017; or Bollard et al., Blood, Vol. 32, No. 8, 2014, each of which is incorporated by reference in its entirety. In certain embodiments, the TCR recognizes a tumor antigen (including a TAA or TSA). In certain embodiments, the TCR is expressed in a tumor-specific T cell. In certain embodiments, the tumor-specific T cell is a tumor-infiltrating T cell generated by culturing T cells with explants of a tumor, e.g., melanoma or an epithelial cancer. In certain embodiments, the tumor-specific T cell is a T cell disclosed in Stevanovic et al, Science, 356, 200-205, 2017; Dudley et al. Journal of Immunotherapy, 26 (4): 332-342, 2003; or Goff et al, Journal of Clinical Oncology, Vol. 34, No. 20, 2016, each of which is incorporated by reference in its entirety.

### 5.4.3. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARS. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD35 chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In accordance with the presently disclosed subject matter, a CAR comprises an extracellular antigen-binding domain, a transmembrane domain, and an intracellular signaling domain, wherein the extracellular antigen-binding domain specifically binds to an antigen, e.g., a tumor antigen (TAA or TSA) or a pathogen antigen.

In certain embodiments, the CAR comprises an extracellular antigen-binding domain that binds to CD19, a transmembrane domain, and an intracellular signaling domain.

### 5.4.3.1. Extracellular Antigen-Binding Domain of a CAR

The extracellular antigen-binding domain of the CAR binds to an antigen. The subject matter of the instant application, e.g., cells comprising a FasL polypeptide, a gene disruption of a Fas locus, and expression of an antigen-recognizing receptor, finds use irrespective of the particular antigen bound by the antigen-recognizing receptor. For example, in certain embodiments, the antigen is a tumor antigen. In certain embodiments, the antigen is a pathogen antigen. In certain embodiments, the extracellular antigen-binding domain is a single chain variable fragment (scFv). In certain embodiments, the scFv is a human scFv. In certain embodiments, the scFv is a humanized scFv. In certain embodiments, the scFv is a murine scFv. In certain embodiments, the extracellular antigen-binding domain of the CAR is a Fab, which is optionally crosslinked. In certain embodiments, the extracellular antigen-binding domain of the CAR is a F(ab)₂. In certain embodiments, any of the foregoing molecules may be comprised in a fusion protein with a heterologous sequence to form the extracellular antigen-binding domain.

In certain embodiments, the extracellular antigen-binding domain of the CAR (for example, an scFv) binds to the first antigen with a dissociation constant (K_{d}) of about 1×10⁻⁷ M or less, about 1×10⁻⁸ M or less, or about 1×10⁻⁹M or less, or about 1×10⁻¹⁰ M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

### 5.4.3.2. Transmembrane Domain of a CAR

In certain embodiments, the CAR comprises a transmembrane domain. In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the antigen-recognizing receptor can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the antigen-recognizing receptor (e.g., a CAR) comprises a CD28 polypeptide (e.g., the transmembrane domain of CD28 or a portion thereof). In certain embodiments, the CD28 polypeptide comprises or consists of an amino acid sequence that is a consecutive portion of the amino acid sequence having a NCBI Reference No: NP_006130 (SEQ ID NO: 16), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 16. In certain embodiments, the transmembrane domain of the antigen-recognizing receptor (e.g., a CAR) comprises a CD28 polypeptide that comprises or consists of amino acids 153 to 179 of SEQ ID NO: 16. SEQ ID NO: 16 is provided below.

[SEQ ID NO: 16]
MLRLLLALNLEPSIQVTGNKILVKQSPMLVAYDNAVNLSCKYSYNLESR
EFRASLHKGLDSAVEVCVVYGNYSQQLQVYSKTGENCDGKLGNESVTFY
LQNLYVNQTDIYFCKIEVMYPPPYLDNEKSNGTIIHVKGKHLCPSPLEP
GPSKPFWVLVVVGGVLACYSLLVTVAFIIFWVRSKRSRLLHSDYMNMTP
RRPGPTRKHYQPYAPPRDFAAYRS

### 5.4.3.3. Hinge/Spacer Region of the CAR

In certain embodiments, the CAR comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 16), a portion of a CD8 polypeptide, a variation of any of the foregoing which is at least about 80%, at least about 85%, at least about 90%, at least about 95%, or at least about 100% homologous or identical thereto, or a synthetic spacer sequence.

In certain embodiments, the antigen-recognizing receptor is a CAR that further comprises a hinge/spacer region comprising a native or modified hinge region of a CD28 polypeptide. In certain embodiments, the hinge/spacer region of the antigen-recognizing receptor (e.g., a CAR) comprises a CD28 polypeptide comprising or consisting of amino acids 114 to 152 of SEQ ID NO: 16.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

### 5.4.3.4. Intracellular Signaling Domain of a CAR

In certain embodiments, the CAR comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3ζ transmits an activation signal to the cell (e.g., a cell of the lymphoid lineage, e.g., a T-cell) after antigen is bound. The intracellular signaling domain of the CD3ζ-chain is the primary transmitter of signals from endogenous TCRs.

In certain embodiments, the intracellular signaling domain of the antigen-recognizing receptor comprises a native CD3ζ. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 17) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 17, which is at least about 20, or at least about 30, or at least about 40, or at least about 50, and up to about 164 amino acids in length. In certain embodiments, the native CD3ζ comprises or consists of the amino acid sequence of amino acids 1 to 164, 1 to 50, 50 to 100, 52 to 164, 100 to 150, or 150 to 164 of SEQ ID NO: 17. In certain embodiments, the intracellular signaling domain of the CAR comprises a native CD3ζ comprising or consisting of the amino acid sequence of amino acids 52 to 164 of SEQ ID NO: 17. SEQ ID NO: 17 is provided below:

[SEQ ID NO: 17]
MKWKALFTAAILQAQLPITEAQSFGLLDPKLCYLLDGILFIYGVILTAL
FLRVKESRSADAPAYQQGONQLYNELNLGRREEYDVLDKRRGRDPEMGG
KPQRRKNPQEGLYNELQKDKMAEAYSEIGMKGERRRGKGHDGLYQGLST
ATKDTYDALHMQALPPR

In certain embodiments, the native CD3ζ comprises or consists of an amino acid sequence that is at least about 85%, about 90%, about 95%, about 96%, about 97%, about 98%, about 99% or about 100% identical or homologous to the amino acid sequence set forth in SEQ ID NO: 18. SEQ ID NO: 18 is provided below:

[SEQ ID NO: 18]
RVKESRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 19.

[SEQ ID NO: 19]
QNQLYNELNLGRREEYDVLDKR

An exemplary nucleic acid sequence encoding the amino acid sequence of SEQ ID NO: 19 is set forth in SEQ ID NO: 20, which is provided below.

[SEQ ID NO: 20]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 21, which is provided below.

[SEQ ID NO: 21]
QNQLENELNLGRREEFDVLDKR

An exemplary nucleic acid sequence encoding the amino acid sequence of SEQ ID NO: 21 is set forth in SEQ ID NO: 22, which is provided below.

[SEQ ID NO: 22]
CAGAACCAGCTCTTTAACGAGCTCAATCTAGGACGAAGAGAGGAGTTC
GATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 23, which is provided below.

[SEQ ID NO: 23]
QEGLYNELOKDKMAEAYSEIGMK

An exemplary nucleic acid sequence encoding the amino acid sequence of SEQ ID NO: 23 is set forth in SEQ ID NO: 24, which is provided below.

[SEQ ID NO: 24]
CAGGAAGGCCTGTACAATGAACTGCAGAAAGATAAGATGGCGGAGGCC
TACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 25, which is provided below.

[SEQ ID NO: 25]
QEGLFNELQKDKMAEAFSEIGMK

An exemplary nucleic acid sequence encoding the amino acid sequence of SEQ ID NO: 25 is set forth in SEQ ID NO: 26, which is provided below.

[SEQ ID NO: 26]
CAGGAAGGCCTGTTCAATGAACTGCAGAAAGATAAGATGGCGGAGGCCTT
CAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 27, which is provided below.

[SEQ ID NO: 27]
HDGLYQGLSTATKDTYDALHMQ

An exemplary nucleic acid sequence encoding the amino acid sequence of SEQ ID NO: 27 is set forth in SEQ ID NO: 28, which is provided below.

[SEQ ID NO: 28]
CACGATGGCCTTTACCAGGGTCTCAGTACAGCCACCAAGGACACCTACGA
CGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 29, which is provided below. HDGLFQGLSTATKDTFDALHMQ [SEQ ID NO: 29]

An exemplary nucleic acid sequence encoding the amino acid sequence of SEQ ID NO: 29 is set forth in SEQ ID NO: 30, which is provided below.

[SEQ ID NO: 30]
CACGATGGCCTTTTCCAGGGGCTCAGTACAGCCACCAAGGACACCTTCGA
CGCCCTTCACATGCAG

Various modified CD3ζ polypeptides and CARs comprising modified CD3 polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 19, an ITAM2 variant consisting of the amino acid sequence set forth in SEQ ID NO: 25, and an ITAM3 variant consisting of the amino acid sequence set forth in SEQ ID NO: 29. In certain embodiments, the CAR is designated as “1XX”. In certain embodiments, the modified CD3ζ polypeptide comprises or consists of the amino acid sequence set forth in SEQ ID NO: 31. SEQ ID NO: 31 is provided below:

[SEQ ID NO: 31]
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
EDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 31 or a fragment thereof, and/or may optionally comprise up to one or up to two or up to three conservative amino acid substitutions.

An exemplary nucleic acid sequence encoding the amino acid sequence of SEQ ID NO: 31 is set forth in SEQ ID NO: 32, which is provided below.

[SEQ ID NO: 32]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTTCAATGAACTGCAGAAAGATAAGAT
GGCGGAGGCCTTCAGTGAGATTGGGATGAAAGGCGAGCGCCGGAGGGGCA
AGGGGCACGATGGCCTTTTCCAGGGGCTCAGTACAGCCACCAAGGACACC
TTCGACGCCCTTCACATGCAGGCCCTGCCCCCTCGC

In certain embodiments, the intracellular signaling domain of the antigen-recognizing receptor further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, and NKG2D.

In certain embodiments, the intracellular signaling domain of the antigen-recognizing receptor (e.g., a CAR) comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the antigen-recognizing receptor comprise or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 16 or a fragment thereof, and/or may optionally comprise up to one or up to two or up to three conservative amino acid substitutions. In certain embodiments, the CD28 polypeptide comprised in the co-stimulatory signaling region comprises or consist of an amino acid sequence that is a consecutive portion of SEQ ID NO: 16, which is at least about 20, or at least about 30, or at least about 40, or at least about 50, and up to about 220 amino acids in length. Alternatively or additionally, in certain embodiments, the CD28 polypeptide comprised in the co-stimulatory signaling region comprises or consists of amino acids 1 to 220, 1 to 50, 50 to 100, 100 to 150, 114 to 220, 150 to 200, 180 to 220, or 200 to 220 of SEQ ID NO: 16. In certain embodiments, the intracellular signaling domain of the antigen-recognizing receptor comprises a co-stimulatory signaling region that comprises a CD28 polypeptide comprising or consisting of the amino acid sequence of amino acids 180 to 220 of SEQ ID NO: 16.

An exemplary nucleic acid sequence encoding the amino acid sequence of amino acids 180 to 220 of SEQ ID NO: 16 is set forth in SEQ ID NO: 33, which is provided below.

[SEQ ID NO: 33]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the antigen-recognizing receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 34) or a fragment thereof, and/or may optionally comprise up to one or up to two or up to three conservative amino acid substitutions. In certain embodiments, the CD28 polypeptide comprised in the co-stimulatory signaling region comprises or consists of an amino acid sequence that is a consecutive portion of SEQ ID NO: 34, which is at least about 20, or at least about 30, or at least about 40, or at least about 50, and up to 218 amino acids in length. In certain embodiments, the CD28 polypeptide comprised in the co-stimulatory signaling region comprises or consists of the amino acid sequence of amino acids 1 to 218, 1 to 50, 50 to 100, 100 to 150, 150 to 218, 178 to 218, or 200 to 218 of SEQ ID NO: 34. In certain embodiments, the co-stimulatory signaling region of the antigen-recognizing receptor comprises a CD28 polypeptide that comprises or consists of the amino acid sequence of amino acids 178 to 218 of SEQ ID NO: 34. SEQ ID NO: 34 is provided below.

[SEQ ID NO: 34]
MTLRLLFLALNFFSVQVTENKILVKQSPLLVVDSNEVSLSCRYSYNLLAK
EFRASLYKGVNSDVEVCVGNGNFTYQPQFRSNAEENCDGDEDNETVTERL
WNLHVNHTDIYFCKIEFMYPPPYLDNERSNGTIIHIKEKHLCHTQSSPKL
FWALVVVAGVLFCYGLLVTVALCVIWINSRRNRLLQSDYMNMTPRRPGLT
RKPYQPYAPARDFAAYRP

In certain embodiments, the intracellular signaling domain of the antigen-recognizing receptor comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_001552 (SEQ ID NO: 35) or a fragment thereof, and/or may optionally comprise up to one or up to two or up to three conservative amino acid substitutions. In certain embodiments, the 4-1BB comprised in the co-stimulatory signaling region comprises or consists of an amino acid sequence that is a consecutive portion of SEQ ID NO: 35, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the 4-1BB polypeptide comprised in the co-stimulatory signaling region comprises or consists of the amino acid sequence of amino acids 1 to 255, 1 to 50, 50 to 100, 100 to 150, 150 to 200, or 200 to 255 of SEQ ID NO: 35. In certain embodiments, the co-stimulatory signaling region comprises a 4-1BB polypeptide comprising or consisting of the amino acid sequence of amino acids 214 to 255 of SEQ ID NO: 35. SEQ ID NO: 35 is provided below.

[SEQ ID NO: 35]
MGNSCYNIVA TLLLVLNFER TRSLQDPCSN CPAGTFCDNN RNQICSPCPP NSESSAGGQR
TCDICROCKG VERTRKECSS TSNAECDCTP GFHCLGAGCS MCEQDCKQGQ ELTKKGCKDC
CFGTENDQKR GICRPWTNCS LDGKSVLVNG TKERDVVCGP SPADLSPGAS SVTPPAPARE
PGHSPQIISF FLALTSTALL FLLFELTLRF SVVKRGRKKL LYIFKQPEMR PVQTTQEEDG
CSCRFPEEEE GGCEL

In certain embodiments, the intracellular signaling domain of the antigen-recognizing receptor comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, and NKG2D. In certain embodiments, the intracellular signaling domain of the antigen-recognizing receptor comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

### 5.4.4. TCR Like Fusion Molecules

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule comprises an antigen binding chain that comprises an extracellular antigen-binding domain and a constant domain, wherein the TCR like fusion molecule binds to an antigen in an HLA-independent manner. In certain embodiments, the constant domain comprises a T cell receptor constant region selected from the group consisting of a native or modified TRAC peptide, a native or modified TRBC peptide, a native or modified TRDC peptide, a native or modified TRGC peptide and any variants or functional fragments thereof. In certain embodiments, the constant domain comprises a native or modified TRAC peptide. In certain embodiments, the constant domain comprises a native or modified TRBC peptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv). In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv).

The TCR like fusion molecule can bind to a tumor antigen or a pathogen antigen. In certain embodiments, the TCR like fusion molecule binds to a tumor antigen.

### 5.5. Cells

The presently disclosed subject matter provides cells comprising a FasL polypeptide (e.g., one disclosed in Section 5.2) and a gene disruption of a Fas locus (e.g., one disclosed in Section 5.3). In certain embodiments, the cells further comprise an antigen-recognizing receptor (e.g., one disclosed in Section 5.4). In certain embodiments, the FasL polypeptide is an exogenous FasL polypeptide. In certain embodiments, the FasL polypeptide binds to Fas. Fas-FasL interaction induces apoptosis in cells (e.g., tumor cells and immunoresponsive cells). Thus, the presently disclosed cells comprising a Fas ligand polypeptide and a gene disruption of a Fas locus are capable of lysing cells expressing Fas (e.g., endogenous T cells or NK cells).

In certain embodiments, the gene disruption of a Fas locus is a capable of protecting cells from fratricide killing.

In certain embodiments, the cell is an immunoresponsive cell. In certain embodiments, the cell is a cell of the lymphoid lineage. Cells of the lymphoid lineage produce antibodies, regulate cellular immune system, and detect foreign agents in the blood and cells foreign to the host and the like. Non-limiting examples of cells of the lymphoid lineage include T cells, Natural Killer (NK) cells, B cells, dendritic cells, and stem cells from which lymphoid cells may be differentiated. In certain embodiments, the stem cell is a pluripotent stem cell (e.g., embryonic stem cell or induced pluripotent stem cell).

In certain embodiments, the cell is a T cell. T cells can be lymphocytes that mature in the thymus and are chiefly responsible for cell-mediated immunity. T cells are part of the adaptive immune system. In certain embodiments, the T cells provided herein comprise any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells or T_{regs}), tumor-infiltrating lymphocytes (TILs), natural killer T cells, mucosal associated invariant T cells, and γδ T cells. Cytotoxic T cells (CTLs or killer T cells) are a subset of T lymphocytes capable of inducing the death of infected somatic or tumor cells. A patient's own T cells (i.e., autologous T cells) may be genetically modified to target specific antigens through the introduction of an antigen-recognizing receptor, e.g., a CAR or a TCR. In certain embodiments, the cell is a T cell. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a virus-specific T cell. In certain embodiments, the virus-specific T cell comprises an endogenous TCR that recognizes a viral antigen. In certain embodiments, the cell is a tumor-specific T cell. In certain embodiments, the tumor-specific T cell comprises an endogenous TCR that recognizes a tumor antigen (TSA or TAA).

In certain embodiments, the cell is an NK cell. Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells. In certain embodiments, the cell is allogeneic.

In certain embodiments, the cell is a cell of the myeloid lineage. Non-limiting examples of cells of the myeloid lineage include monocytes, macrophages, basophils, neutrophils, eosinophils, mast cell, erythrocytes, megakaryocytes, thrombocytes, and stem cells from which myeloid cells may be differentiated. In certain embodiments, the stem cell is a pluripotent stem cell (e.g., embryonic stem cell or induced pluripotent stem cell).

### 5.5.1. GENE DISRUPTION OF GENETIC LOCI

In certain embodiments, the presently disclosed cell further comprises a gene disruption of a TCR locus. Non-limiting examples of TCR loci include a TRAC locus, a TRBC locus, a TRDC locus, a TRGC locus, or a combination thereof. In certain embodiments, the gene disruption of the TCR locus results in a non-functional T cell receptor. In certain embodiments, the gene disruption of the TCR locus results in knockout of the gene expression of TCRα, TCRβ, TCRγ, TCRδ, or a combination thereof. Any methods disclosed in Section 5.3 can be used to generate the gene disruption of the TCR locus. In certain embodiments, the gene disruption of the TCR locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the TCR locus can be a disruption of the coding region of the TRAC locus and/or a disruption of the non-coding region of the TRAC locus. In certain embodiments, the gene disruption of the TRAC locus comprises a disruption of the coding region of the TRAC locus. In certain embodiments, the gene disruption of the TRAC locus comprises an insertion at the coding region of the TRAC locus. Human TRAC protein comprises four exons: exon 1, exon 2, exon 3, and exon 4. In certain embodiments, the gene disruption of the TRAC locus comprises a disruption at one or more of exon 1, exon 2, exon 3, and exon 4 of the TRAC locus. In certain embodiments, the gene disruption of the TRAC locus comprises a disruption at exon 1 of the TRAC locus. In certain embodiments, the gene disruption of the TRAC locus comprises an insertion at exon 1 of the TRAC locus.

In certain embodiments, the gene disruption of the TCR locus can be a disruption of the coding region of the TRBC locus and/or a disruption of the non-coding region of the TRBC locus. In certain embodiments, the gene disruption of the TRBC locus comprises a disruption of the coding region of the TRBC locus. In certain embodiments, the gene disruption of the TRBC locus comprises an insertion at the coding region of the TRBC locus. Human TRBC protein comprises four exons: exon 1, exon 2, exon 3, and exon 4. In certain embodiments, the gene disruption of the TRBC locus comprises a disruption at one or more of exon 1, exon 2, exon 3, and exon 4 of the TRBC locus. In certain embodiments, the gene disruption of the TRBC locus comprises a disruption at exon 1 of the TRBC locus. In certain embodiments, the gene disruption of the TRBC locus comprises an insertion at exon 1 of the TRBC locus.

In certain embodiments, the gene disruption of the TCR locus can be a disruption of the coding region of the TRDC locus and/or a disruption of the non-coding region of the TRDC locus. In certain embodiments, the gene disruption of the TRDC locus comprises a disruption of the coding region of the TRDC locus. In certain embodiments, the gene disruption of the TRDC locus comprises an insertion at the coding region of the TRDC locus. Human TRDC protein comprises four exons: exon 1, exon 2, exon 3, and exon 4. In certain embodiments, the gene disruption of the TRDC locus comprises a disruption at one or more of exon 1, exon 2, exon 3, and exon 4 of the TRDC locus. In certain embodiments, the gene disruption of the TRDC locus comprises a disruption at exon 1 of the TRDC locus. In certain embodiments, the gene disruption of the TRDC locus comprises an insertion at exon 1 of the TRDC locus.

In certain embodiments, the gene disruption of the TCR locus can be a disruption of the coding region of the TRGC locus and/or a disruption of the non-coding region of the TRGC locus. In certain embodiments, the gene disruption of the TRGC locus comprises a disruption of the coding region of the TRGC locus. In certain embodiments, the gene disruption of the TRGC locus comprises an insertion at the coding region of the TRGC locus. Human TRGC protein comprises three exons: exon 1, exon 2, and exon 3. In certain embodiments, the gene disruption of the TRGC locus comprises a disruption at one or more of exon 1, exon 2, and exon 3 of the TRGC locus. In certain embodiments, the gene disruption of the TRGC locus comprises a disruption at exon 1 of the TRGC locus. In certain embodiments, the gene disruption of the TRGC locus comprises an insertion at exon 1 of the TRGC locus.

In certain embodiments, the cell is a T cell, and the antigen-recognizing receptor (e.g., one disclosed in Section 5.4) is integrated at a TCR locus within the genome of the T cell. In certain embodiments, the cell is a T cell, and the antigen-recognizing receptor is integrated at a TRAC locus. Methods of targeting an antigen-recognizing receptor (e.g., a CAR) to a site within the genome of T cell are disclosed in WO2017180989 and Eyquem et al., Nature (2017 Mar. 2); 543 (7643): 113-117, both of which are incorporated by reference in their entireties. In certain embodiments, the cell is a T cell, the antigen-recognizing receptor is a CAR, and the CAR is integrated at a TRAC locus. In certain embodiments, the gene disruption of a TRAC locus results in knockout of a TRAC locus. In certain embodiments, the cell further comprises a gene disruption of a TRBC locus. In certain embodiments, the gene disruption of a TRBC locus results in knockout of a TRBC locus.

In certain embodiments, a presently disclosed cell further comprises a gene disruption of a B2M locus. In certain embodiments, the gene disruption of the B2M locus results in a non-functional beta 2-microglobulin. In certain embodiments, the gene disruption of the B2M locus results in knockout of the B2M gene expression. Any methods disclosed in Section 5.3 can be used to generate the gene disruption of the B2M locus. In certain embodiments, the gene disruption of the B2M locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the B2M locus can be a disruption of the coding region of the B2M locus and/or a disruption of the non-coding region of the B2M locus. In certain embodiments, the gene disruption of the B2M locus comprises a disruption of the coding region of the B2M locus. In certain embodiments, the gene disruption of the B2M locus comprises an insertion at the coding region of the B2M locus. Human B2M protein comprises four exons: exon 1, exon 2, exon 3, and exon 4. In certain embodiments, the gene disruption of the B2M locus comprises a disruption at one or more of exon 1, exon 2, exon 3, and exon 4 of the B2M locus. In certain embodiments, the gene disruption of the B2M locus comprises a disruption at exon 1 of the B2M locus. In certain embodiments, the gene disruption of the B2M locus comprises an insertion at exon 1 of the B2M locus.

Immune rejection of stem cells is due to the expression of human leucocyte antigen class I molecules (HLA-I) on the surface of these cells (Zhang et al., J Cell Mol Med. (2020); 24:695-710). HLA-I presents “non-self” antigens to CD8+ T cells that eliminate the transplanted cells through direct cytotoxic effect (Zhang 2020). Due to the polymorphic nature of the HLA-I genes, it is often difficult to identify a perfect match between donor and recipient prior to transplantation (Zhang 2020). HLA-I comprises a heavy chain and a light chain, which is also called β2-microglobulin (B2M). HLA-I structure is disrupted and non-functional when the B2M gene is deleted (Zhang 2020). In certain embodiments, the gene disruption of the B2M locus can reduce immune rejection, thereby making the cells more suitable for an allogeneic setting.

In certain embodiments, a presently disclosed cell further comprises a gene disruption of a Class II transactivator (CIITA) locus. In certain embodiments, the gene disruption of the CIITA locus results in a non-functional MHC class II transactivator.

In certain embodiments, the gene disruption of the CIITA locus results in knockout of the CIITA gene expression. Any methods disclosed in Section 5.3 can be used to generate the gene disruption of the CIITA locus. In certain embodiments, the gene disruption of the CIITA locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, the gene disruption of the CIITA locus can be a disruption of the coding region of the CIITA locus and/or a disruption of the non-coding region of the CIITA locus. In certain embodiments, the gene disruption of the CIITA locus comprises a disruption of the coding region of the CIITA locus. In certain embodiments, the gene disruption of the CIITA locus comprises an insertion at the coding region of the CIITA locus. Human CIITA protein comprises 22 exons: exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, and exon 22. In certain embodiments, the gene disruption of the CIITA locus comprises a disruption at one or more of exon 1 through exon 22 of the CIITA locus. In certain embodiments, the gene disruption of the CIITA locus comprises a disruption at exon 3 of the CIITA locus. In certain embodiments, the gene disruption of the B2M locus comprises an insertion at exon 3 of the CIITA locus.

CIITA is a transcriptional coactivator that regulates γ-interferon-activated transcription of Major Histocompatibility Complex (MHC) class I and II genes (Devaiah et al., Frontiers in Immunology (2013); Vol. 4; Article 476:1-6). Thus, CIITA plays a critical role in immune responses: CIITA deficiency results in aberrant MHC gene expression and consequently in autoimmune diseases such as Type II bare lymphocyte syndrome (Devaiah 2013). Although CIITA does not bind to DNA directly, it regulates MHC transcription in two distinct ways—as a transcriptional activator and as a general transcription factor (Devaiah 2013). The CIITA is a master regulator of MHC gene expression (Devaiah 2013). CIITA induces de novo transcription of MHC class II genes and enhances constitutive MHC class I gene expression (Devaiah 2013). MHC II expression is regulated by CIITA. In certain embodiments, the gene disruption of the CIITA locus can reduce immune rejection, and improve survival of allogeneic bone marrow stem cells, thereby making the cells more suitable for an allogeneic setting.

### 5.5.2. Exemplified Cells

In certain embodiments, the cell comprises an antigen-recognizing receptor. In certain embodiments, the antigen-recognizing receptor is a CAR. In certain embodiments, the cell comprises a FasL polypeptide, and a gene disruption of a Fas locus. In certain embodiments, the gene disruption of the Fas locus results in knockout of Fas. In certain embodiments, the cell is a T cell. In certain embodiments, the cell is a NK cell.

In certain embodiments, the cell comprises an antigen-recognizing receptor. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the cell further comprises a gene disruption of a TRAC locus. In certain embodiments, the gene disruption of the TRAC locus results in knockout of the TRAC locus. In certain embodiments, the cell is a T cell. In certain embodiments, the cell is a NK cell.

In certain embodiments, the cell comprises an antigen-recognizing receptor. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the cell further comprises a gene disruption of a TRAC locus. In certain embodiments, the gene disruption of the TRAC locus results in knockout of the TRAC locus. In certain embodiments, the cell further comprises a gene disruption of a TRBC locus. In certain embodiments, the gene disruption of the TRBC locus results in knockout of the TRBC locus. In certain embodiments, the cell is a T cell. In certain embodiments, the cell is a NK cell.

In certain embodiments, the cell comprises an antigen-recognizing receptor. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the cell further comprises a gene disruption of a TRAC locus. In certain embodiments, the gene disruption of the TRAC locus results in knockout of the TRAC locus. In certain embodiments, the cell comprises a gene disruption of a B2M locus. In certain embodiments, the gene disruption of the B2M locus results in knockout of the B2M locus. In certain embodiments, the cell is a T cell. In certain embodiments, the cell is a NK cell.

In certain embodiments, the cell comprises an antigen-recognizing receptor. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the cell further comprises a gene disruption of a TRAC locus. In certain embodiments, the gene disruption of the TRAC locus results in knockout of the TRAC locus. In certain embodiments, the cell further comprises a gene disruption of a B2M locus. In certain embodiments, the gene disruption of the B2M locus results in knockout of the B2M locus. In certain embodiments, the cell further comprises a gene disruption of a CIITA locus. In certain embodiments, the gene disruption of the CIITA locus results in knockout of the CIITA locus. In certain embodiments, the cell is a T cell. In certain embodiments, the cell is a NK cell.

In certain embodiments, the cell comprises an antigen-recognizing receptor. In certain embodiments, the antigen-recognizing receptor is integrated in a TRAC locus. In certain embodiments, the antigen-recognizing receptor is a CAR. In certain embodiments, the cell comprises a FasL polypeptide and a Fas locus. In certain embodiments, the cell is a T cell. In certain embodiments, the cell is a NK cell.

In certain embodiments, the cell comprises an antigen-recognizing receptor. In certain embodiments, the antigen-recognizing receptor is integrated in a TRAC locus. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the cell further comprises a gene disruption of a B2M locus. In certain embodiments, the gene disruption of the B2M locus results in knockout of the B2M locus. In certain embodiments, the cell is a T cell. In certain embodiments, the cell is a NK cell.

In certain embodiments, the cell comprises an antigen-recognizing receptor. In certain embodiments, the antigen-recognizing receptor is integrated in a TRAC locus. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the cell further comprises a gene disruption of a TRBC locus. In certain embodiments, the gene disruption of the TRBC locus results in knockout of the TRBC locus. In certain embodiments, the cell is a T cell. In certain embodiments, the cell is a NK cell.

In certain embodiments, the cell comprises an antigen-recognizing receptor. In certain embodiments, the antigen-recognizing receptor is integrated in a TRAC locus. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the cell further comprises a gene disruption of a B2M locus. In certain embodiments, the gene disruption of the B2M locus results in knockout of the B2M locus. In certain embodiments, the cell further comprises a gene disruption of a CIITA locus. In certain embodiments, the gene disruption of the CIITA locus results in knockout of the CIITA locus. In certain embodiments, the cell is a T cell. In certain embodiments, the cell is a NK cell.

In certain embodiments, the cell comprises an antigen-recognizing receptor. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the antigen-recognizing receptor and the FasL polypeptide are integrated in a TRAC locus. In certain embodiments, the cell is a T cell. In certain embodiments, the cell is a NK cell.

In certain embodiments, the cell comprises an antigen-recognizing receptor. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the antigen-recognizing receptor and the FasL polypeptide are integrated in a TRAC locus. In certain embodiments, the cell further comprises a gene disruption of a B2M locus. In certain embodiments, the gene disruption of the B2M locus results in knockout of the B2M locus. In certain embodiments, the cell is a T cell. In certain embodiments, the cell is a NK cell.

In certain embodiments, the cell comprises an antigen-recognizing receptor. In certain embodiments, the antigen-recognizing receptor is a CAR. In certain embodiments, the cell comprises a FasL polypeptide and a gene disruption of a Fas locus. In certain embodiments, the gene disruption of the Fas locus results in knockout of Fas. In certain embodiments, the antigen-recognizing receptor and the FasL polypeptide are integrated in a TRAC locus. In certain embodiments, the cell further comprises a gene disruption of a B2M locus. In certain embodiments, the gene disruption of the B2M locus results in knockout of the B2M locus. In certain embodiments, the cell further comprises a gene disruption of a CIITA locus. In certain embodiments, the gene disruption of the CIITA locus results in knockout of the CIITA locus. In certain embodiments, the cell is a T cell. In certain embodiments, the cell is a NK cell.

### 5.6. Nucleic Acid Compositions and Vectors

The presently disclosed subject matter provides nucleic acid compositions comprising a first polynucleotide encoding a FasL polypeptide disclosed herein (e.g., disclosed in Section 5.2). In certain embodiments, the nucleic acid compositions further comprise a second polynucleotide encoding an antigen-recognizing receptor disclosed herein (e.g., disclosed in Section 5.4). Also provided are cells comprising such nucleic acid compositions.

In certain embodiments, the first polynucleotide is operably linked to a first promoter. In certain embodiments, the second polynucleotide is operably linked to a second promoter.

In certain embodiments, the first and/or the second promoters are endogenous or exogenous. Non-limiting examples of exogenous promoters include an elongation factor (EF)-1 promoter, a CMV promoter, a SV40 promoter, a PGK promoter, a long terminal repeat (LTR) promoter and a metallothionein promoter. Non-limiting examples of inducible promoters include a NFAT transcriptional response element (TRE) promoter, a CD69 promoter, a CD25 promoter, an IL-2 promoter, an IL-12 promoter, a p40 promoter, and a Bcl-xL promoter.

In certain embodiments, the FasL polypeptide and/or the antigen-recognizing receptors are integrated at a locus within the genome of the T cell, e.g., a TRAC locus, a TRBC locus, a TRDC locus, or a TRGC locus. In certain embodiments, the locus is a TRAC locus. In certain embodiments, the expression of the FasL polypeptide and/or the antigen-recognizing receptors are under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

The presently disclosed subject matter provides vectors comprising the presently disclosed nucleic acid compositions. In certain embodiments, the vector is a retroviral vector. In certain embodiments, the vector is a lentiviral vector or a gamma-retroviral vector.

The compositions and nucleic acid compositions can be administered to subjects and/or delivered into cells by methods known in the art or as described herein. Genetic modification of a cell (e.g., a T cell) can be accomplished by transducing a substantially homogeneous cell composition with a recombinant DNA construct. In certain embodiments, a retroviral vector (either a gamma-retroviral vector or a lentiviral vector) is employed for the introduction of the DNA construct into the cell. Non-viral vectors may be used as well.

For initial genetic modification of a cell to include a FasL polypeptide, and optionally an antigen-recognizing receptor, a retroviral vector is generally employed for transduction, however any other suitable viral vector or non-viral delivery system can be used. The FasL polypeptide, and optionally the antigen-recognizing receptor can be constructed in a single, multicistronic expression cassette, in multiple expression cassettes of a single vector, or in multiple vectors. Examples of elements that can be used to create a multicistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides).

Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107: 77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to deliver the FasL polypeptide disclosed herein. In certain embodiments, the same methods can also be used to deliver the antigen-recognizing receptor disclosed herein to a cell or a subject. In certain embodiments, a CRISPR system is used to deliver the FasL polypeptide disclosed herein. In certain embodiments, a CRISPR system is used also to deliver the antigen-recognizing receptor disclosed herein. In certain embodiments, zinc-finger nucleases are used to deliver the FasL polypeptide disclosed herein. In certain embodiments, zinc-finger nucleases are also used to deliver the antigen-recognizing receptor disclosed herein. In certain embodiments, a TALEN system is used to deliver the FasL polypeptide disclosed herein. In certain embodiments, a TALEN system is used to deliver the antigen-recognizing receptor disclosed herein.

The clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALENs) are restriction enzymes that can be engineered to cut specific sequences of DNA. A TALENs system operates on almost the same principle as ZFNs. They are generated by combining a transcription activator-like effectors DNA-binding domain with a DNA cleavage domain. Transcription activator-like effectors (TALEs) comprise 33-34 amino acid repeating motifs with two variable positions that have a strong recognition for specific nucleotides. By assembling arrays of these TALEs, the TALE DNA-binding domain can be engineered to bind a desired DNA sequence, and thereby guide the nuclease to cut at specific locations in genomic DNA sequences.

Polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

The resulting cells can be grown under conditions similar to those for unmodified cells, whereby the modified cells can be expanded and used for a variety of purposes.

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3″-(((methylazanediyl) bis(propane-3,1 diyl))bis(azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9″,9″ “,9”-((((benzene-1,3,5-tricarbonyl)yris(azanediyl))tris(propane-3,1-diyl))tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9″Z,12Z, 12′Z, 12″Z, 12″Z)-tetrakis(octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 5.7. Formulations and Administration

The presently disclosed subject matter also provides compositions comprising the presently disclosed cells (e.g., those disclosed in Section 5.5). Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The presently disclosed cells can be administered in any physiologically acceptable vehicle, normally intravascularly, although they may also be introduced into bone or other convenient site where the cells may find an appropriate site for regeneration and differentiation (e.g., thymus).

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, 5×10⁵, 1×10⁶, about 5×10⁶, about 1×10⁷, about 5×10⁷, about 1×10⁸, or about 5×10⁸ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

In certain embodiments, the composition is a pharmaceutical composition comprising the presently disclosed cells and a pharmaceutically acceptable carrier.

Administration of the compositions can be autologous or heterologous. For example, cells can be obtained from one subject, and administered to the same subject or a different, compatible subject. Peripheral blood derived cells or their progeny (e.g., in vivo, ex vivo or in vitro derived) can be administered. When administering a presently disclosed composition (e.g., a pharmaceutical composition comprising presently disclosed cells), it can be formulated in a unit dosage injectable form (solution, suspension, emulsion).

The presently disclosed cells and compositions can be administered by any method known in the art including, but not limited to, oral administration, intravenous administration, portal vein administration, subcutaneous administration, intranodal administration, intratumoral administration, intrathecal administration, intracranial administration, intrapleural administration, intraosseous administration, intraperitoneal administration, pleural administration, intrabronchial arteries administration, intralesional administration, and direct administration to the subject. In certain embodiments, the presently disclosed cells and compositions can be administered by hepatic artery pump.

### 5.8. Methods of Treatment

The presently disclosed subject matter provides various methods of using the presently disclosed cells or compositions comprising thereof. The presently disclosed subject matter provides methods for inducing lysis of a target cell expressing Fas. In certain embodiments, the method comprises administering to the target cell the presently disclosed cells or composition comprising thereof. The presently disclosed cells comprise a FasL polypeptide, which binds to Fas. The Fas-FasL interaction induces apoptosis in cells (e.g., tumor cells and immunoresponsive cells). Thus, the presently disclosed cells comprising a FasL polypeptide are capable of lysing the target cells expressing Fas. Importantly, the Fas-FasL-mediated lysis is capable of inducing lysis of immune cells (e.g., host T cells and/or host NK cells).

In certain embodiments, the target cells comprise tumor cells. In certain embodiments, the target cells comprise immunoresponsive cells. In certain embodiments, the target cells is a cell of the lymphoid lineage. In certain embodiments, the target cell is a T cell. In certain embodiments, the T cells comprises any type of T cells, including, without any limitation, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells or T_{regs}), tumor-infiltrating lymphocytes (TILs), natural killer T cells, mucosal associated invariant T cells, and γδ T cells. In certain embodiments, the T cell can be a CD4⁺ T cell or a CD8⁺ T cell. In certain embodiments, the T cell is a CD4⁺ T cell. In certain embodiments, the T cell is a CD8⁺ T cell. In certain embodiments, the target cell is a NK cell.

In certain embodiments, the target cell is an endogenous cell. In certain embodiments, the target cell is a host cell.

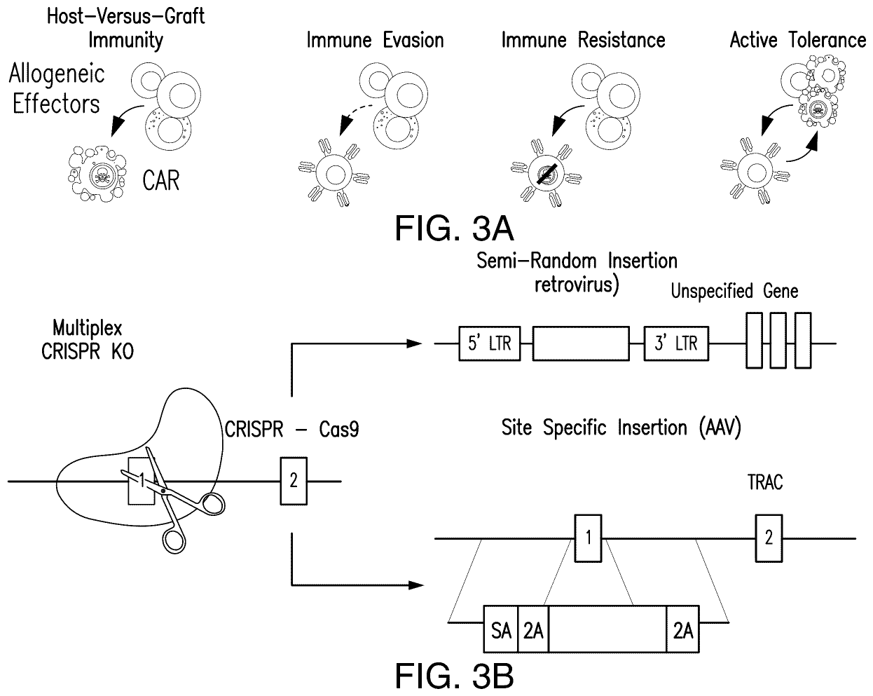

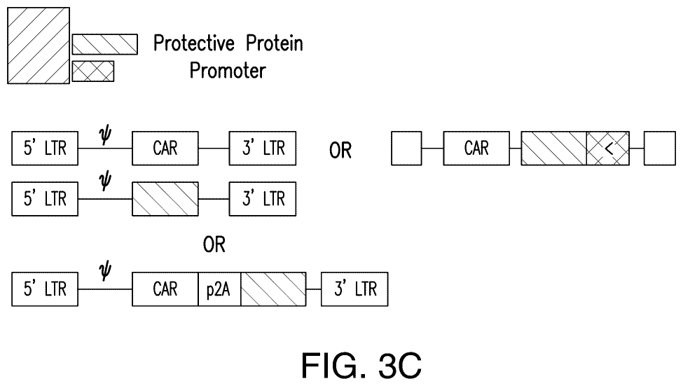

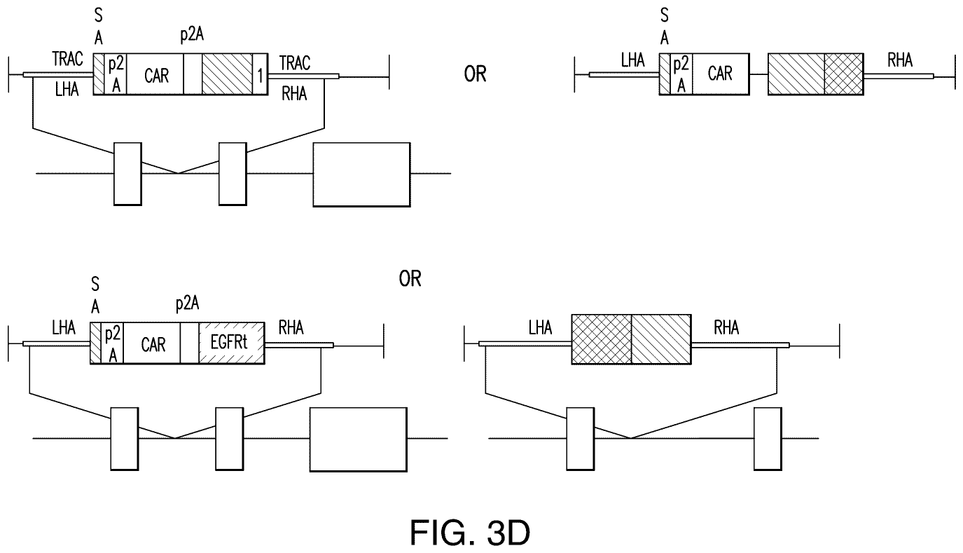

The gene disruption of a Fas locus is capable of protecting the presently disclosed cells from fratricide killing and from host versus graft reactions. In allogeneic settings, host-versus-graft immunity can lead to rejection of the presently disclosed cells or composition comprising thereof. As depicted in FIG. 3A, the presently disclosed cells or composition comprising thereof are resistant to host-versus-graft immunity because they are resistant to cell death caused by allogencic immunity (e.g., host T cells or NK cells). Furthermore, the presently disclosed cells or composition comprising thereof can induce active immune tolerance by deletion of alloreactive immune effector cells (e.g., host T cells or NK cells) via Fas-FasL-mediated apoptosis. Thus, the presently disclosed cells can lyse target cells expressing Fas, while the presently disclosed cells themselves can be protected by the gene disruption of a Fas locus.

The presently disclosed subject matter further provides methods for treating a disease or disorder in a subject. Non-limiting examples of diseases or disorders include tumors, pathogen infections, autoimmune diseases, and infectious diseases. In certain embodiments, the methods comprise administering to a subject suffering from a disease or disorder the presently disclosed cells or a composition comprising the cells. Since the Fas-FasL-mediated lysis is antigen-independent, the presently disclosed cells can be used for treating diseases or disorders that are not responsive well to antigen-dependent therapies due to lack of targetable antigens.

In certain embodiments, the disease or disorder is a tumor. In certain embodiments, the presently disclosed cells or composition can reduce tumor burden, induce tumor cell death, reduce the number of tumor cells, reduce tumor size, and/or eradicate the tumor in the subject.

In certain embodiments, the tumor is a solid tumor. In solid tumor immunotherapy, lack of a targetable antigen that is uniformly overexpressed on cancer cells can be overcome by antigen-independent cytotoxic strategies.

In certain embodiments, the tumor is a hematological tumor. Non-limiting examples of hematological tumors include leukemias and lymphomas (e.g., Hodgkin lymphoma, non-Hodgkin's lymphoma, and B-cell lymphomas). In certain embodiments, the tumor is cancer. In certain embodiments, the tumor is hematological malignancy. Non-limiting examples of tumors include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, and B cell prolymphocytic leukemia. Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm (e.g., malignant neoplasm) is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer (including non-small cell lung cancer (NSCLC) and small cell lung cancer (SCLC), pancreatic cancer, prostate cancer, skin cancer, stomach cancer, glioblastoma, and throat cancer.

Non-limiting examples of solid tumors include glioblastoma, prostate adenocarcinoma, kidney papillary cell carcinoma, sarcoma, ovarian cancer, pancreatic adenocarcinoma, rectum adenocarcinoma, colon adenocarcinoma, esophageal carcinoma, uterine corpus endometrioid carcinoma, breast cancer, skin cutaneous melanoma, non-small cell lung cancer (NSCLC), lung adenocarcinoma, stomach adenocarcinoma, cervical and endocervical cancer, kidney clear cell carcinoma, and testicular germ cell tumors.

In certain embodiments, the disease or disorder is a pathogen infection or an infectious disease. In certain embodiments, the method comprises administering to a subject suffering from a pathogen infection or an infectious disease the presently disclosed cells comprising the FasL polypeptide, the gene disruption of a Fas locus, and an antigen-recognizing receptor (e.g., a CAR) that binds to a pathogen antigen (e.g., one disclosed in Section 5.4.1). In certain embodiments, the disease or disorder is an autoimmune disease.

In certain embodiments, the method further comprises administering to the subject a second therapy. In certain embodiments, the second therapy comprises cyclophosphamide preconditioning, radiation therapy, chemotherapy, an adoptive cell therapy, a therapy comprising an immune checkpoint inhibitor, or a combination thereof. Non-limiting examples of adoptive cell therapies include therapies comprising immunoresponsive cell comprising a chimeric antigen receptor, immunoresponsive cells comprising a T cell receptor, and immunoresponsive cells comprising a T cell receptor like fusion molecule. Non-limiting examples of immune checkpoint inhibitors include anti-PD-L1 antibodies, anti-CTLA-4 antibodies, anti-PD-1 antibodies, anti-LAG3 antibodies, anti-B7-H3 antibodies, anti-TIM3 antibodies, anti-TIGIT antibodies, anti-LAIR1 antibodies, anti-2B4 antibodies, and anti-CD160 antibodies. In certain embodiments, the immune checkpoint inhibitor is an anti-PD-L1 antibody or an anti-PD-1 antibody. The second therapy can be administered to the subject prior to, contemporaneously, or post the administration of the presently disclosed cells or composition. In certain embodiments, the second therapy is administered prior to the administration of the presently disclosed cells or composition. In certain embodiments, the subject is a human.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated receptor-expressing (e.g., CAR-expressing) T cells. The incorporation of a suicide gene into an antigen-recognizing receptor (e.g., a CAR) gives an added level of safety with the ability to eliminate the majority of receptor-expressing (e.g., CAR-expressing) T cells within a very short time period. A presently disclosed cell incorporated with a suicide gene can be pre-emptively eliminated at a given timepoint post T cell infusion, or eradicated at the earliest signs of toxicity.

### 5.9. Kits

The presently disclosed subject matter provides kits for inducing and/or enhancing an immune response in a subject, treating and/or preventing a tumor or neoplasm in a subject, reducing tumor burden in a subject, and/or increasing or lengthening survival of a subject having a tumor or neoplasm in a subject. In certain embodiments, the kit comprises the presently disclosed cells or a composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain embodiments, the kit includes nucleic acid compositions comprising nucleic acid compositions disclosed herein (e.g., disclosed in Section 5.6). In certain embodiments, the kit includes vectors comprising nucleic acid compositions disclosed herein (e.g., disclosed in Section 5.6).

If desired, the cells and/or nucleic acid molecules are provided together with instructions for administering the cells or nucleic acid molecules to a subject having or at risk of developing a tumor or neoplasm. The instructions generally include information about the use of the composition for the treatment and/or prevention of a tumor or neoplasm. In certain embodiments, the instructions include at least one of the following: description of the therapeutic agent; dosage schedule and administration for treatment or prevention of a tumor or neoplasm; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 5.10. Exemplary Embodiments

A1. In certain non-limiting embodiments, the presently disclosed subject matter provides an immunoresponsive cell comprising a) an antigen-recognizing receptor that targets an antigen; b) an exogenous Fas ligand polypeptide (FasL); and c) a gene disruption of a Fas locus.

A2. The foregoing immunoresponsive cell of A1, wherein the FasL polypeptide is capable of binding to Fas.

A3. The foregoing immunoresponsive cell of A1 or A2, wherein the FasL polypeptide is membrane bound.

A4. The foregoing immunoresponsive cell of A3, wherein the FasL polypeptide comprises or consists of an amino acid sequence that is at least about 80% identical to the amino acid sequence set forth in SEQ ID NO: 10.

A5. The foregoing immunoresponsive cell of A3 or A4, wherein the FasL polypeptide comprises or consists of an amino acid sequence set forth in SEQ ID NO: 10.

A6. The foregoing immunoresponsive cell of A3, wherein the FasL polypeptide comprises or consists of an amino acid sequence that is at least about 80% identical to the amino acid sequence set forth in SEQ ID NO: 12.

A7. The foregoing immunoresponsive cell of A3 or A6, wherein the FasL polypeptide comprises or consists of the amino acid sequence set forth in SEQ ID NO: 12.

A8. The foregoing immunoresponsive cell of A3, wherein the FasL polypeptide comprises or consists of an amino acid sequence that is at least about 80% identical to the amino acid sequence set forth in SEQ ID NO: 36.

A9. The foregoing immunoresponsive cell of A3 or A8, wherein the FasL polypeptide comprises or consists of the amino acid sequence set forth in SEQ ID NO: 36.

A10. The foregoing immunoresponsive cell of A3, wherein the FasL polypeptide comprises or consists of an amino acid sequence that is at least about 80% identical to the amino acid sequence set forth in SEQ ID NO: 38.

All. The foregoing immunoresponsive cell of A3 or A10, wherein the FasL polypeptide comprises or consists of the amino acid sequence set forth in SEQ ID NO: 38.

A12. The foregoing immunoresponsive cell of A3, wherein the FasL polypeptide comprises or consists of an amino acid sequence that is at least about 80% identical to the amino acid sequence set forth in SEQ ID NO: 40.

A13. The foregoing immunoresponsive cell of A3 or A12, wherein the FasL polypeptide comprises or consists of the amino acid sequence set forth in SEQ ID NO: 40.

A14. The foregoing immunoresponsive cell of A3, wherein the FasL polypeptide comprises or consists of an amino acid sequence that is at least about 80% identical to the amino acid sequence set forth in SEQ ID NO: 42.

A15. The foregoing immunoresponsive cell of A3 or A14, wherein the FasL polypeptide comprises or consists of the amino acid sequence set forth in SEQ ID NO: 42.

A16. The foregoing immunoresponsive cell of A3, wherein the FasL polypeptide comprises a truncated intracellular domain.

A17. The foregoing immunoresponsive cell of A3, wherein the FasL polypeptide does not comprise an intracellular domain.

A18. The foregoing immunoresponsive cell of A3 or A17, wherein the FasL polypeptide comprises or consists of an amino acid sequence of amino acids 81 to 281 of SEQ ID NO: 9.

A19. The foregoing immunoresponsive cell of A3 or A17, wherein the FasL polypeptide comprises or consists of an amino acid sequence of amino acids 81 to 277 of SEQ ID NO: 12.

A20. The foregoing immunoresponsive cell of any one of A1-A19, wherein the FasL polypeptide is expressed from a vector.

A21. The foregoing immunoresponsive cell of any one of A1-A20, wherein the gene disruption comprises a mutation, a substitution, a deletion, an insertion, or a combination thereof.

A22. The foregoing immunoresponsive cell of A21, wherein the mutation comprises a missense mutation, a nonsense mutation, or a combination thereof.

A23. The foregoing immunoresponsive cell of A21, wherein the deletion comprises a non-frameshift deletion, a frameshift deletion, or a combination thereof.

A24. The foregoing immunoresponsive cell of A21, wherein the insertion comprises a non-frameshift insertion, a frameshift insertion, or a combination thereof.

A25. The foregoing immunoresponsive cell of any one of A1-A24, wherein the gene disruption of the Fas locus results in a non-functional Fas protein.

A26. The foregoing immunoresponsive cell of any one of A1-A25, wherein the gene disruption of the Fas locus results in knockout of the Fas gene expression.

A27. The foregoing immunoresponsive cell of any one of A1-A26, wherein the gene disruption of the Fas locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

A28. The foregoing immunoresponsive cell of any one of A1-A27, wherein the antigen-recognizing receptor is a recombinant T cell receptor (TCR), a chimeric antigen receptor (CAR), or a TCR like fusion molecule.

A29. The foregoing immunoresponsive cell of A28, wherein the antigen-recognizing receptor is a CAR.

A30. The foregoing immunoresponsive cell of A28 or A29, wherein the antigen-recognizing receptor is encoded by a polynucleotide inserted into a first locus within the genome.

A31. The foregoing immunoresponsive cell of A30, wherein the first locus is selected from the group consisting of a TRAC locus, a TRBC locus, a TRDC locus, a TRGC locus, and a Fas locus.

A32. The foregoing immunoresponsive cell of A30 or A31, wherein the first locus is a Fas locus.

A33. The foregoing immunoresponsive cell of A30 or A31, wherein the first locus is a TRAC locus.

A34. The foregoing immunoresponsive cell of any one of A1-A33, wherein the FasL polypeptide is encoded by a polynucleotide inserted into a second locus within the genome.

A35. The foregoing immunoresponsive cell of A34, wherein the second locus is selected from the group consisting of a TRAC locus, a TRBC locus, a TRDC locus, a TRGC locus, and a Fas locus.

A36. The foregoing immunoresponsive cell of A34 or A35, wherein the second locus is a Fas locus.

A37. The foregoing immunoresponsive cell of A34 or A35, wherein the second locus is a TRAC locus.

A38. The foregoing immunoresponsive cell of any one of A1-A37, wherein the antigen-recognizing receptor and the FasL polypeptide are encoded by a polynucleotide inserted into a first locus within the genome.

A39. The foregoing immunoresponsive cell of A38, wherein the first locus is selected from the group consisting of a TRAC locus, a TRBC locus, a TRDC locus, a TRGC locus, and a Fas locus.

A40. The foregoing immunoresponsive cell of A38 or A39, wherein the first locus is a TRAC locus.

A41. The foregoing immunoresponsive cell of any one of A1-A40, wherein the antigen is a tumor antigen or a pathogen antigen.

A42. The foregoing immunoresponsive cell of any one of A1-A41, wherein the antigen is a tumor antigen.

A43. The foregoing immunoresponsive cell of A42, wherein the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, and ERBB.

A44. The foregoing immunoresponsive cell of any one of A1-A43, further comprising a gene disruption of a TRAC locus, a TRBC locus, a TRDC locus, and a TRGC locus, or a combination thereof.

A45. The foregoing immunoresponsive cell of A44, wherein the gene disruption comprises a substitution, a deletion, an insertion, or a combination thereof.

A46. The foregoing immunoresponsive cell of A44 or A45, wherein the gene disruption results in a non-functional protein or in knockout of the gene expression.

A47. The foregoing immunoresponsive cell of any one of A44-A46, wherein the gene disruption is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

A48. The foregoing immunoresponsive cell of any one of A1-A47, wherein the cell further comprises a gene disruption of a B2M locus.

A49. The foregoing immunoresponsive cell of A48, wherein the gene disruption of the B2M locus results in a non-functional beta 2-microglobulin.

A50. The foregoing immunoresponsive cell of A48 or A49, wherein the gene disruption of the B2M locus results in knockout of the B2M gene expression.

A51. The foregoing immunoresponsive cell of any one of A48-A50, wherein the gene disruption of the B2M locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

A52. The foregoing immunoresponsive cell of any one of A1-A51, wherein the cell further comprises a gene disruption of a CIITA locus.

A53. The foregoing immunoresponsive cell of A52, wherein the gene disruption of the CIITA locus results in a non-functional MHC class 11 transactivator.

A54. The foregoing immunoresponsive cell of A52 or a53, wherein the gene disruption of the CIITA locus results in knockout of the CIITA gene expression.

A55. The foregoing immunoresponsive cell of any one of A52-A54, wherein the gene disruption of the CIITA locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

A56. The foregoing immunoresponsive cell of any one of A1-A55, wherein the gene disruption of the Fas locus is capable of enhancing at least one activity of the cell comprising the antigen-recognizing receptor.

A57. The foregoing immunoresponsive cell of A56, wherein the at least one activity comprises cytotoxicity, cell proliferation, cell persistence, or a combination thereof

A58. The foregoing immunoresponsive cell of any one of A1-A57, wherein the immunoresponsive cell is a cell of the lymphoid lineage or a cell of the myeloid lineage.

A59. The foregoing immunoresponsive cell of any one of A1-A58, wherein the cell is selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof.

A60. The foregoing immunoresponsive cell of any one of A1-A59, wherein the cell is a T cell.

A61. The foregoing immunoresponsive cell of any one of A1-A59, wherein the cell is a Natural Killer (NK) cell.

A62. The foregoing immunoresponsive cell of any one of A1-A61, wherein the cell is autologous.

A63. The foregoing immunoresponsive cell of any one of A1-A61, wherein the cell is allogeneic.

B1. In certain embodiments, the presently disclosed subject matter provides a composition comprising the immunoresponsive cell of any one of A1-A63.

B2. The foregoing composition of B1, which is a pharmaceutical composition further comprising a pharmaceutically acceptable excipient.

C1. A method for producing a cell of any one of A1-A63, the method comprising:
a) generating a gene disruption of a Fas locus in a cell; and
b) introducing into the cell a polynucleotide encoding a FasL polypeptide.

C2. The foregoing method of C1, further comprising c) introducing into the cell a polynucleotide encoding an antigen-recognizing receptor.

C3. The foregoing method of C1 or C2, wherein the gene disruption comprises a mutation, a substitution, a deletion, an insertion, or a combination thereof.

C4. The foregoing method of C3, wherein the mutation comprises a missense mutation, a nonsense mutation, or a combination thereof.

C5. The foregoing method of C3, wherein the deletion comprises a non-frameshift deletion, a frameshift deletion, or a combination thereof.

C6. The foregoing method of C3, wherein the insertion comprises a non-frameshift insertion, a frameshift insertion, or a combination thereof.

C7. The foregoing method of any one of C1-C6, wherein the gene disruption of the Fas locus results in a non-functional Fas protein.

C8. The foregoing method of any one of C1-C6, wherein the gene disruption of the Fas locus results in knockout of the Fas gene expression.

C9. The foregoing method of any one of C1-C8, wherein generating the gene disruption of the Fas locus comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

C10. The foregoing method of any one of C1-C9, wherein the FasL polypeptide comprises an amino acid sequence that is at least about 80% identical to the amino acid sequence set forth in SEQ ID NO: 10.

C11. The foregoing method of C10, wherein the FasL polypeptide comprises the amino acid sequence set forth in SEQ ID NO: 10.

C12. The foregoing method of any one of C1-C9, wherein the FasL polypeptide comprises an amino acid sequence that is at least about 80% identical to the amino acid sequence set forth in SEQ ID NO: 12.

C13. The foregoing method of C12, wherein the FasL polypeptide comprises the amino acid sequence set forth in SEQ ID NO: 12.

C14. The foregoing method of any one of C1-C9, wherein the FasL polypeptide comprises or consists of an amino acid sequence that is at least about 80% identical to the amino acid sequence set forth in SEQ ID NO: 36.

C15. The foregoing method of C14, wherein the FasL polypeptide comprises or consists of the amino acid sequence set forth in SEQ ID NO: 36.

C16. The foregoing method of any one of C1-C9, wherein the FasL polypeptide comprises or consists of an amino acid sequence that is at least about 80% identical to the amino acid sequence set forth in SEQ ID NO: 38.

C17. The foregoing method of C16, wherein the FasL polypeptide comprises or consists of the amino acid sequence set forth in SEQ ID NO: 38.

C18. The foregoing method of any one of C1-C9, wherein the FasL polypeptide comprises or consists of an amino acid sequence that is at least about 80% identical to the amino acid sequence set forth in SEQ ID NO: 40.

C19. The foregoing method of C18, wherein the FasL polypeptide comprises or consists of the amino acid sequence set forth in SEQ ID NO: 40.

C20. The foregoing method of any one of C1-C9, wherein the FasL polypeptide comprises or consists of an amino acid sequence that is at least about 80% identical to the amino acid sequence set forth in SEQ ID NO: 42.

C21. The foregoing method of C20, wherein the FasL polypeptide comprises or consists of the amino acid sequence set forth in SEQ ID NO: 42.

C22. The foregoing method of any one of C1-C9, wherein the FasL polypeptide comprises a truncated intracellular domain.

C23. The foregoing method of any one of C1-C9, wherein the FasL polypeptide does not comprise an intracellular domain.

C24. The foregoing method of C23, wherein the FasL polypeptide comprises or consists of an amino acid sequence of amino acids 81 to 281 of SEQ ID NO: 9.

C25. The foregoing method of C23, wherein the FasL polypeptide comprises or consists of an amino acid sequence of amino acids 81 to 277 of SEQ ID NO: 12.

C26. The foregoing method of any one of C1-C25, further comprising generating a gene disruption of a TRAC locus in the cell.

C27. The foregoing method of C26, wherein the gene disruption of the TRAC locus results in a non-functional T cell receptor (TCR).

C28. The foregoing method of C26, wherein the gene disruption of the TRAC locus results in knockout of the TCR gene expression.

C29. The foregoing method of any one of C26-C28, wherein generating the gene disruption of the TRAC locus to the cell comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

C30. The foregoing method of any one of C1-C29, further comprising generating a gene disruption of a B2M locus in the cell.

C31. The foregoing method of C30, wherein the gene disruption of the B2M locus results in a non-functional beta 2-microglobulin.

C32. The foregoing method of C30, wherein the gene disruption of the B2M locus results in knockout of the B2M gene expression.

C33. The foregoing method of any one of C30-C32, wherein generating the gene disruption of the B2M locus to the cell comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

C34. The foregoing method of any one of C1-C33, further comprising generating a gene disruption of a CIITA locus in the cell.

C35. The foregoing method of C34, wherein the gene disruption of the CIITA locus results in a non-functional MHC class II transactivator.

C36. The foregoing method of C34, wherein the gene disruption of the CIITA locus results in knockout of the CIITA gene expression.

C37. The foregoing method of any one of C34-C36, wherein generating the gene disruption of the CIITA locus to the cell comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

D1. In certain non-limiting embodiments, the presently disclosed subject matter provides an immunoresponsive cell produced by the method of any one of C1-C37.

E1. In certain non-limiting embodiments, the presently disclosed subject matter provides a nucleic acid composition comprising a first polynucleotide encoding a Fas ligand polypeptide, and a second polynucleotide encoding a nuclease.

E2. The foregoing nucleic acid composition of E1, wherein the FasL polypeptide comprises or consists of an amino acid sequence that is at least about 80% identical to the amino acid sequence set forth in SEQ ID NO: 10, SEQ ID NO: 12, SEQ ID NO: 36, SEQ ID NO: 38, SEQ ID NO: 40, or SEQ ID NO: 42.

E3. The foregoing nucleic acid composition of E1 or E2, wherein the FasL polypeptide comprises or consists of an amino acid sequence set forth in SEQ ID NO: 10, SEQ ID NO: 12, SEQ ID NO: 36, SEQ ID NO: 38, SEQ ID NO: 40, or SEQ ID NO: 42.

E4. The foregoing nucleic acid composition of E1, wherein the FasL polypeptide comprises a truncated intracellular domain.

E5. The foregoing nucleic acid composition of E1, wherein the FasL polypeptide does not comprise an intracellular domain.

E6. The foregoing nucleic acid composition of E5, wherein the FasL polypeptide comprises or consists of an amino acid sequence of amino acids 81 to 281 of SEQ ID NO: 9.

E7. The foregoing nucleic acid composition of E5, wherein the FasL polypeptide comprises or consists of an amino acid sequence of amino acids 81 to 277 of SEQ ID NO: 12.

E8. The foregoing nucleic acid composition of any one of E1-E7, wherein the nuclease is selected from the group consisting of a Zinc finger nuclease, a meganuclease, a Transcription activator-like effector nuclease (TALEN), and a Cas nuclease.

E9. The foregoing nucleic acid composition of any one of E1-E8, further comprising a third polynucleotide comprising a gRNA.

E10. The foregoing nucleic acid composition of E9, wherein the gRNA comprises or consists of the nucleotide sequence set forth in SEQ ID NO: 15.

E11. The foregoing nucleic acid composition of any one of E1-E10, further comprising a fourth polynucleotide encoding an antigen-recognizing receptor that binds to an antigen.

E12. The foregoing nucleic acid composition of E11, wherein the antigen-recognizing receptor is a TCR, a CAR, or a TCR like fusion molecule.

E13. The foregoing nucleic acid composition of E11 or E12, wherein the antigen-recognizing receptor is a CAR.

F1. In certain non-limiting embodiments, the presently disclosed subject matter provides a lipid nanoparticle comprising the nucleic acid composition of any one of E1-E13.

G1. In certain non-limiting embodiments, the presently disclosed subject matter provides an immunoresponsive cell comprising the nucleic acid composition of any one of E or the lipid nanoparticle of F1.

H1. In certain non-limiting embodiments, the presently disclosed subject matter provides a method of lysing a target cell expressing Fas, comprising contacting the target cell with the cell of any one of A1-A63, D1 or G1, or the composition of B1 or B2.

H2. The foregoing method of H1, wherein the target cell comprises a tumor cell.

H3. The foregoing method of H1 or H2, wherein the target cell comprises an immune cell.

H4. The foregoing method of H3, wherein the immune cell comprises a T cell, a Natural Killer (NK) cell, or a combination thereof.

I1. In certain non-limiting embodiments, the presently disclosed subject matter provides a method of reducing tumor burden in a subject, the method comprising administering to the subject an effective amount of the immunoresponsive cells of any one of A1-A63, D1 or G1, or the composition of B1 or B2.

I2. The foregoing method of I1, wherein the method reduces the number of tumor cells, reduces tumor size, and/or eradicates the tumor in the subject.

I3. In certain non-limiting embodiments, the presently disclosed subject matter provides a method of preventing and/or treating a tumor in the subject, administering to the subject an effective amount of the immunoresponsive cells of any one of A1-A63, D1 or G1, or the composition of B1 or B2.

I4. In certain non-limiting embodiments, the presently disclosed subject matter provides a method of treating a disease or a disorder in a subject, comprising administering to the subject the immunoresponsive cells of any one of A1-A63, D1 or G1, or the composition of B1 or B2.

I5. The foregoing method of I4, wherein the disease or disorder is selected from tumors, pathogen infections, autoimmune diseases, and infectious diseases.

I6. The foregoing method of I4 or I5, wherein the disease or disorder is a tumor.

I7. The foregoing method of I6, wherein the cell or composition reduces tumor burden, induces tumor cell death, reduces the number of tumor cells, reduces tumor size, and/or eradicates the tumor in the subject.

I8. The foregoing method of any one of I1-I7, wherein the tumor is a solid tumor.

I9. The foregoing method of any one of I1-I7, wherein the tumor is a hematological tumor.

I10. The method of any one of I1-I9, wherein the tumor is a cancer.

I11. The foregoing method of I4 or I5, wherein the disease or disorder is a pathogen infection or an infectious disease.

I12. The foregoing method of I4 or I5, wherein the disease or disorder is an autoimmune disease.

I13. The foregoing method of any one of I1-I13, wherein the subject is human.

J1. In certain non-limiting embodiments, the presently disclosed subject matter provides the immunoresponsive cells of any one of A1-A63, D1 or G1, or the composition of B1 or B2 for use in reducing tumor burden in a subject, preventing and/or treating a tumor in the subject, and/or treating a disease or a disorder in a subject.

K1. In certain non-limiting embodiments, the presently disclosed subject matter provides a kit for reducing tumor burden in a subject, treating and/or preventing a tumor in a subject, and/or increasing or lengthening survival of a subject having a tumor, comprising the cell of any one of A1-A63, D1 or G1, or the composition of B1 or B2.

K2. The foregoing kit of K1, wherein the kit further comprises written instructions for using the cell for reducing tumor burden in a subject, treating and/or preventing a tumor or neoplasm in a subject, and/or increasing or lengthening survival of a subject having a tumor

### 6. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1—Multiplex Editing of CAR T Cells

The presently disclosed subject matter demonstrates that gene editing using the CRISPR/Cas9 technology allows generation CAR T cells for use in allogeneic setting.

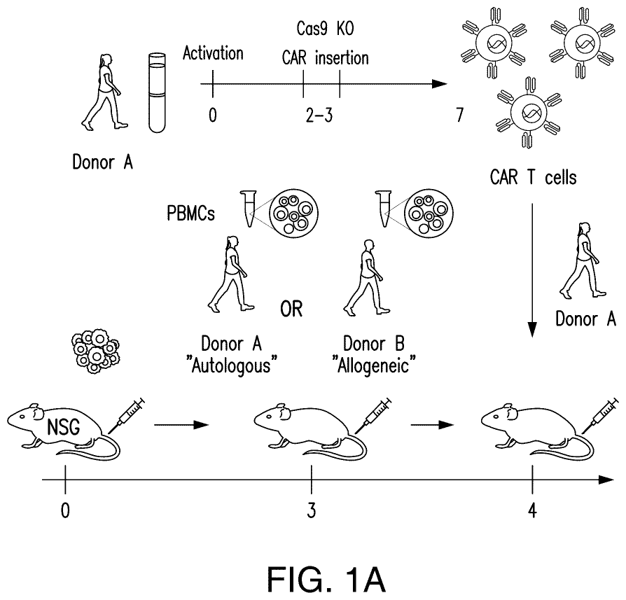

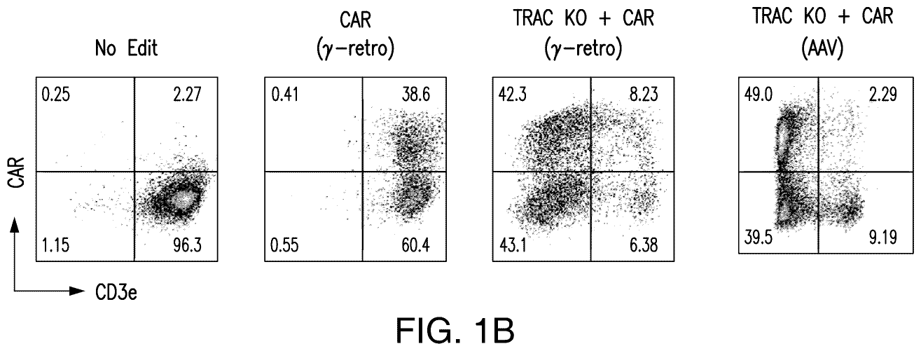

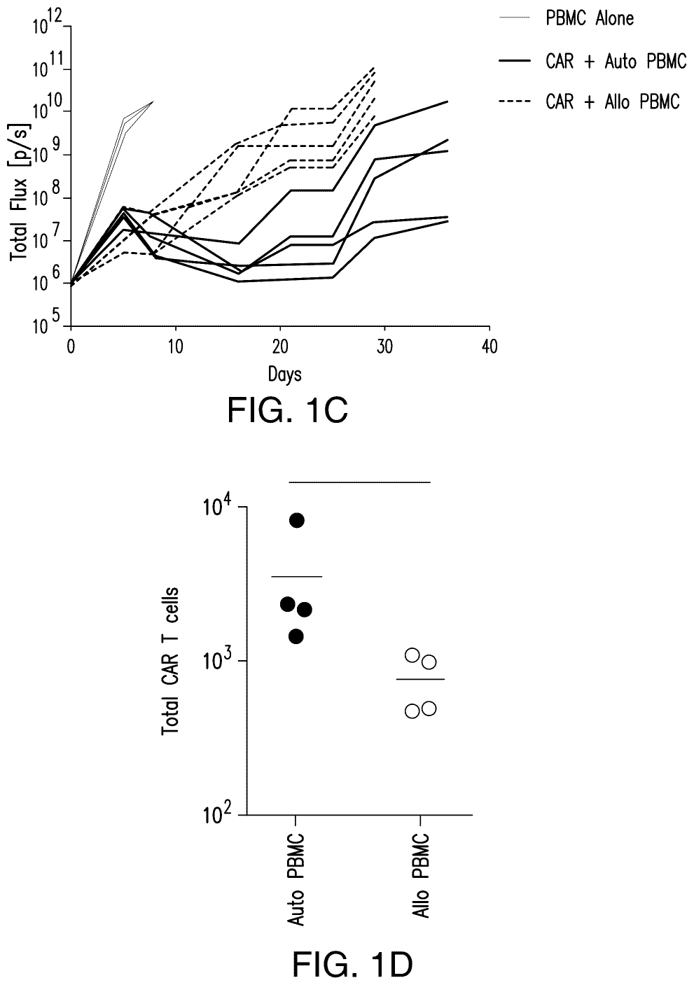

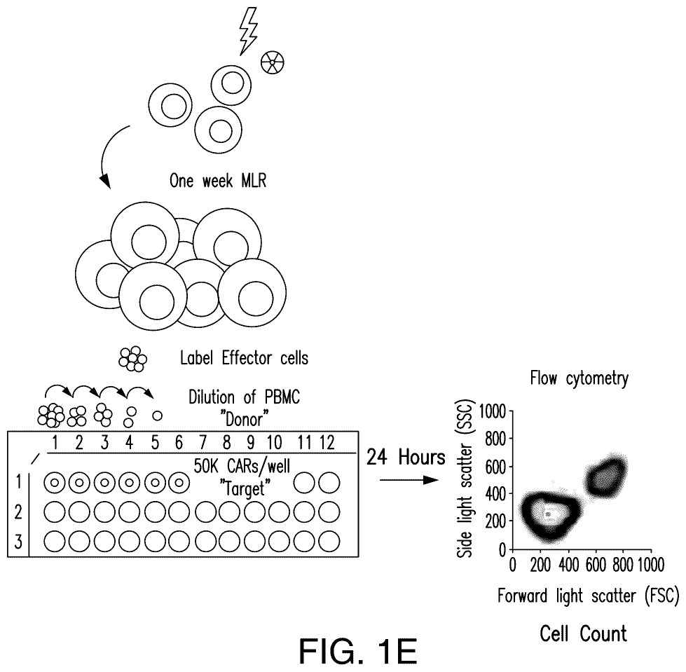

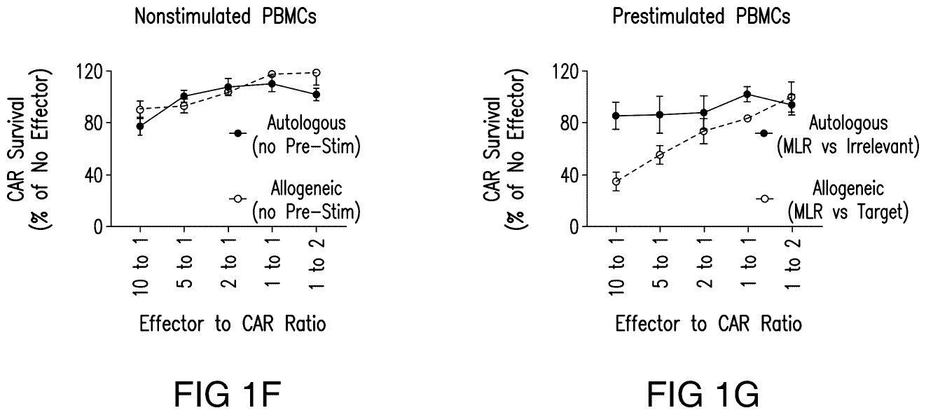

Initially, a mouse model to study the immune rejections of allogeneic CAR T cells was developed. As shown in FIG. 1A, CAR T cells were generated by CRISPR-Cas9 targeting of TRAC locus, followed by CAR insertion via either semi-random gamma-retrovirus mediated transduction, or homology-directed repair driven insertion of CAR into the TRAC locus. NALM6 leukemia cell line transduced with GFP and firefly luciferase for bioluminescent imaging were injected on Day 0. PBMCs from either the same donor (autologous) or a different donor (allogeneic) as CAR T cells were injected intravenously on Day 3, followed by CAR T cells on Day 4. Tumor growth was monitored by BLI.

CAR T cells edited with Cas9 and a TRAC-directed guide RNA showed loss of expression of CD38, which is associated with TCRα. Importantly, CAR T cells generated with either γ-retrovirus or AAV had robust CAR expression (see FIG. 1B). Next, CAR T cells were infused into mice model with autologous or allogeneic PBMCs. As shown in FIG. 1C, CAR T cells infused with autologous PBMCs had superior tumor control to either a PBMC-alone control or CAR T cells infused with allogeneic PBMCs. Notably, fewer CAR T cells were found in bone marrow ten days after CAR injection in mice carrying allogeneic compared to autologous PBMCs. These data indicate that an immune rejection (e.g., host-versus-graft) against CAR T cells was occurring.

To further study and monitoring immune rejection of CAR T cells, an in vitro assay was developed (see FIG. 1E). Briefly, PBMC from Donor B were stimulated with irradiated PBMCs from Donor A (Mixed Leukocyte Reaction, MLR). CAR T cells were generated from Donor A. After one week, CAR T cells were incubated with titrated doses of fluorescently labeled, MLR-stimulated PBMCs and flow cytometry were used to measure CAR T cell survival. CAR T cell survival was unimpaired in the presence of freshly isolated autologous or allogeneic PBMCs. However, when PBMCs were MLR-stimulated for one week, allogeneic PBMCs impaired survival of CAR T cells compared to autologous PBMCs at high effector: target ratios (see FIGS. 1F and 1G).

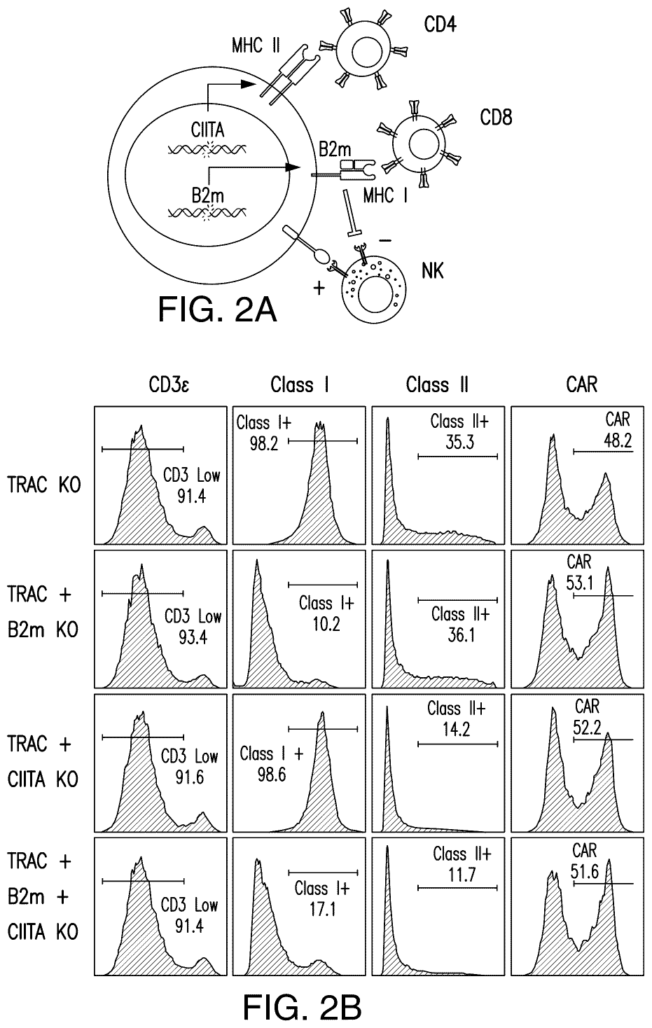

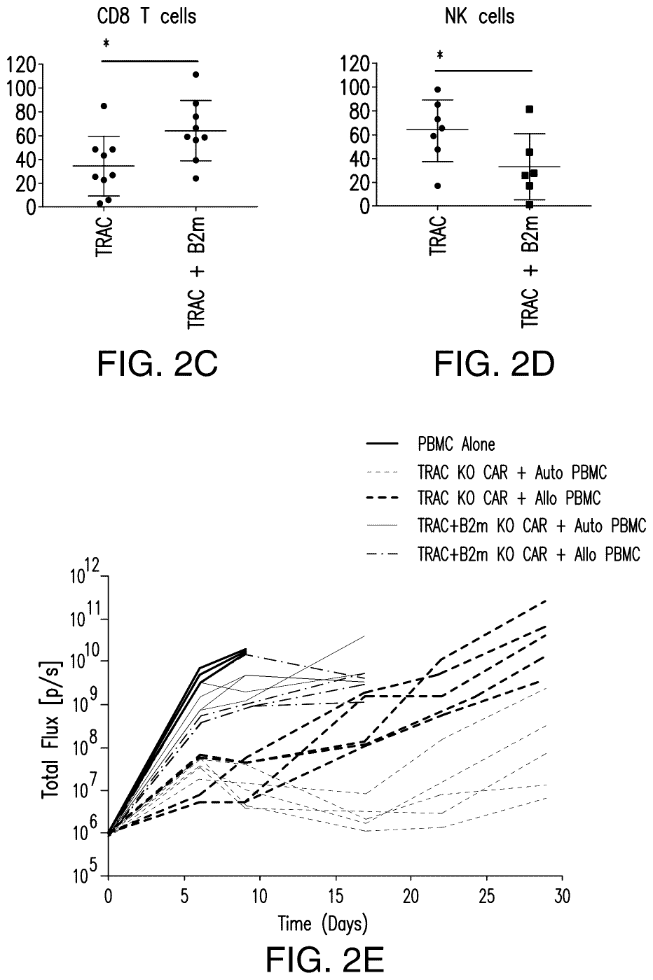

Next, it was determined whether multiple CRISPR-Cas9 editing of CAR T cells could be generated and whether could evade immunity. FIG. 2A shows a schematic of allogeneic host-versus-graft immunity against CAR T cells. CD8 T cells typically recognize antigen via MHC Class I, whereas CD4 T cells typically target antigen via MHC Class II. Thus, expression of Class I and Class II MHC can be modulated by editing of the structural component beta-2-macroglobulin via B2M gene, and transcriptional activator CIITA via CIITA gene.

Multiplex editing targeting 2 or 3 loci followed by CAR transduction was efficient, with about 50% CAR expression and more than 75% of Class I and Class II double-negative among CAR T cells (see FIG. 2B). Notably, CAR T cell lacking MHC Class I via TRAC and B2m editing (TRAC+B2m) or TRAC, B2m, and CIITA (Triple) were protected from CD8 T cell killing (see FIG. 2C) but were sensitive to NK cell killing at a 2:1 effector: target ratio in an in vitro survival assay (see FIG. 2D). Interestingly, in an in vivo model, TRAC KO CAR T cells infused with autologous but not allogeneic PBMCs mediated tumor control, whereas CAR T cells with TRAC and B2m KO were unable to mediate tumor control in either condition. Thus, a more effective allogeneic CAR T cell strategy was developed.

Example 2—Strategies for development of allogeneic CAR T cells

As shown in FIG. 3A, several strategies can be adopted or developed to obtain CAR T cells that are resistant to host-versus-graft immunity. In one strategy, allogeneic CAR T cells can be made undetectable to allogeneic immunity by modulating antigen detection (e.g., evasion). In another strategy, CAR T cells can be resistant to cell death or suppression caused by allogeneic immunity. Alternatively or additionally, CAR T cells can induce active immune tolerance by deletion of alloreactive immune effector cells. Allogeneic resistant CAR T cells can be generated by multiplex CRISPSR-Cas9 editing of target genes, such as TRAC, Fas, or CIITA. CAR and additional gene cassettes can be introduced via either gamma-retroviral transduction or by site specific insertion via homology-driven repair from AAV.

Several vectors can be used for obtaining allogeneic CAR T cells. As shown in FIGS. 3C, the vector can introduce a CAR construct (e.g., 1928z or 1928z-1xx), and can also introduce a protein that confers protection via immune evasion, immune resistance, or active tolerance, such as a FasL polypeptide disclosed herein. CAR and protective proteins can be introduced as separate retroviral vectors or as a single bicistronic construct in a gamma-retrovirus. Alternatively, expression of the protective protein is placed under control of a promoter such as EF1α or PGK or PGK100 in a lentiviral construct.

In another example, AAV vectors can be used for delivering of the constructs. These vectors, as shown in FIG. 3D, can include a left and right homology arm (LHA and RHA, respectively). In some instances, the CAR and the protective protein can be expressed as a single bicistronic vector with homology arms targeting insertion in a single gene, such as TRAC or FAS. The protective protein can be separate using a P2A skip sequence, or place in reverse orientation such that it is under the control of an alternative promoter. In other instances, the CAR and the protective protein can be introduced under separate vectors for specific insertion into separate sites, for example within both the TRAC and FAS genes.

### Example 3—Fas Knockout and FasL Overexpression for Development of Allogeneic CAR T Cells

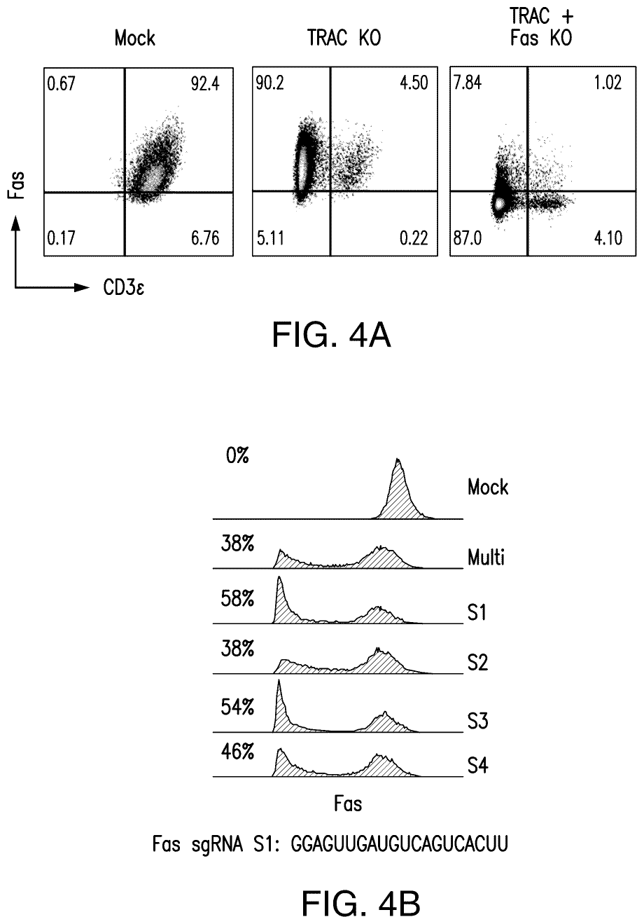

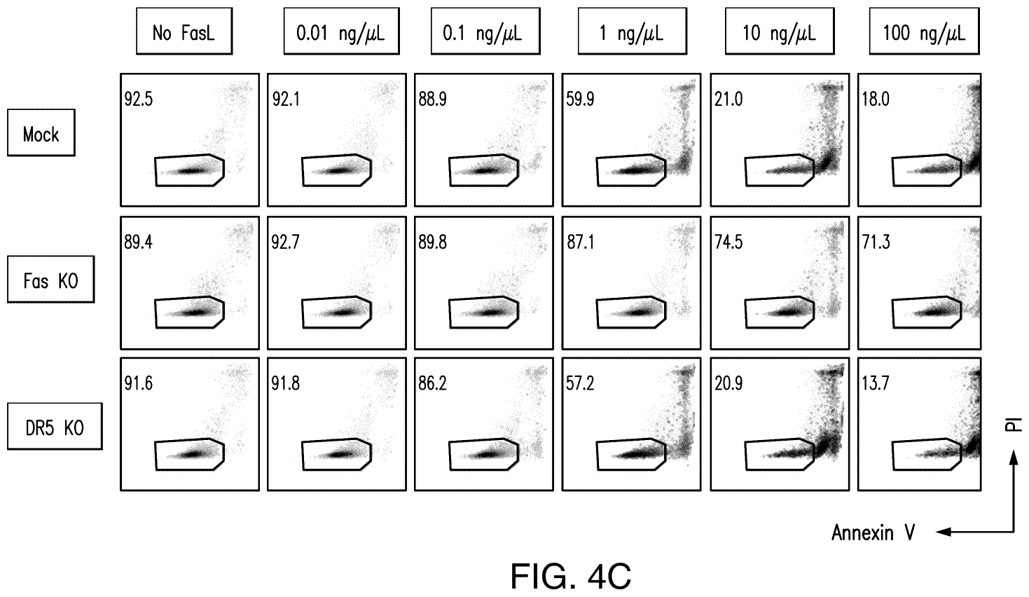

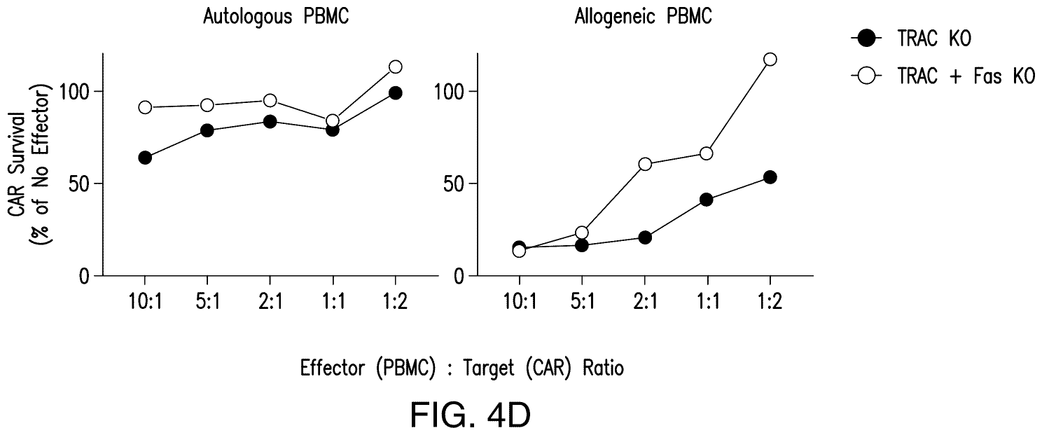

In order to develop allogeneic CAR T cells capable of being resistant to cell death or suppression caused by allogeneic immunity (e.g., host T cells or NK cells), CAR T cells were edited with CRISPR-Cas9 sgRNA targeting either TRAC locus alone or with Fas. As shown in FIG. 4A, knockout of TRAC and Fas induced high percentage of TCR and Fas negative population.

Next, several sgRNAs were screened for targeting Fas locus. As shown in FIG. 4B, four designed RNAs (S1-S4) outperformed a commercially available sgRNA pool (“multi”). Next, it was determined whether Fas knockout (Fas KO) could render CAR T cells less sensitive to recombinant exogenous Fas Ligand. Recombinant exogenous FasL was cross-linked with anti-His antibody at increasing concentrations and apoptosis was measured by Annexin V-Propidium Iodide (PI) uptake after six hours. An irrelevant guide target Death Receptor 5 has no effect compared to mock edited. As shown in FIG. 4C, Fas KO CAR T cells were protected by apoptosis induced by recombinant exogenous FasL.

Next, the survival of edited CAR T cells was studied. TRAC- and Fas-KO CAR T cells had increased survival when compared to TRAC-KO edited CAR T cells upon incubation with amounts of MLR-stimulated allogeneic PBMCs. See FIG. 4D. Notably, there was no survival advantage after 24 hours in the presence of autologous PBMCs.

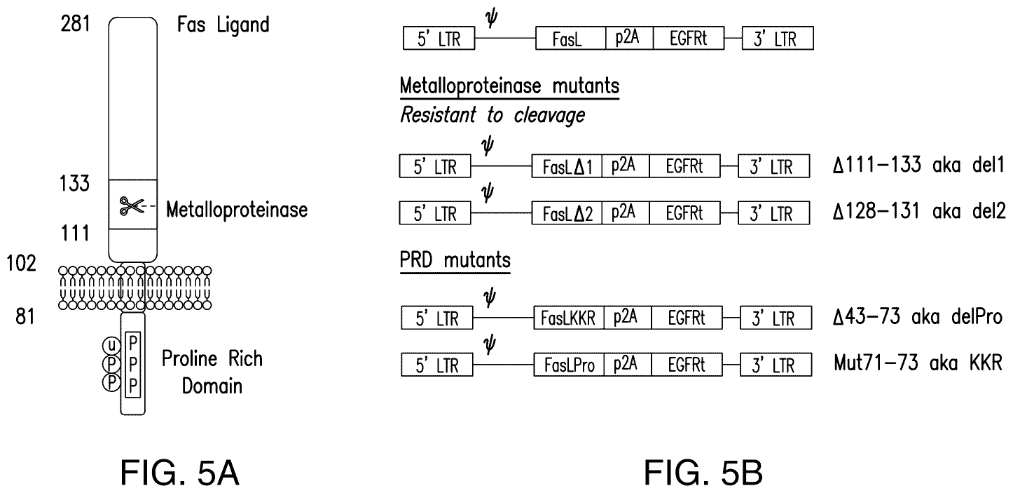

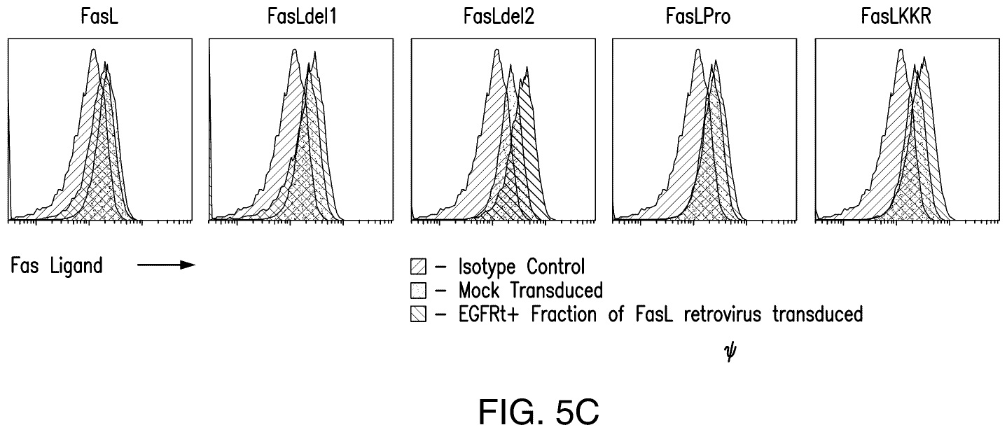

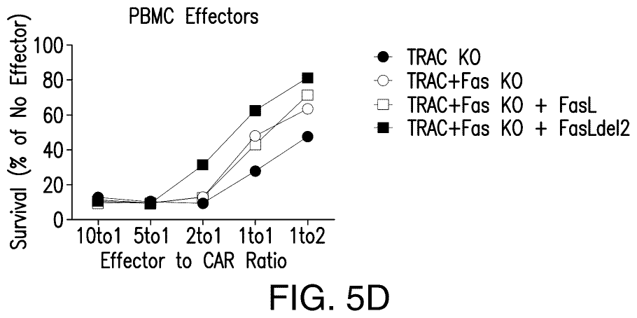

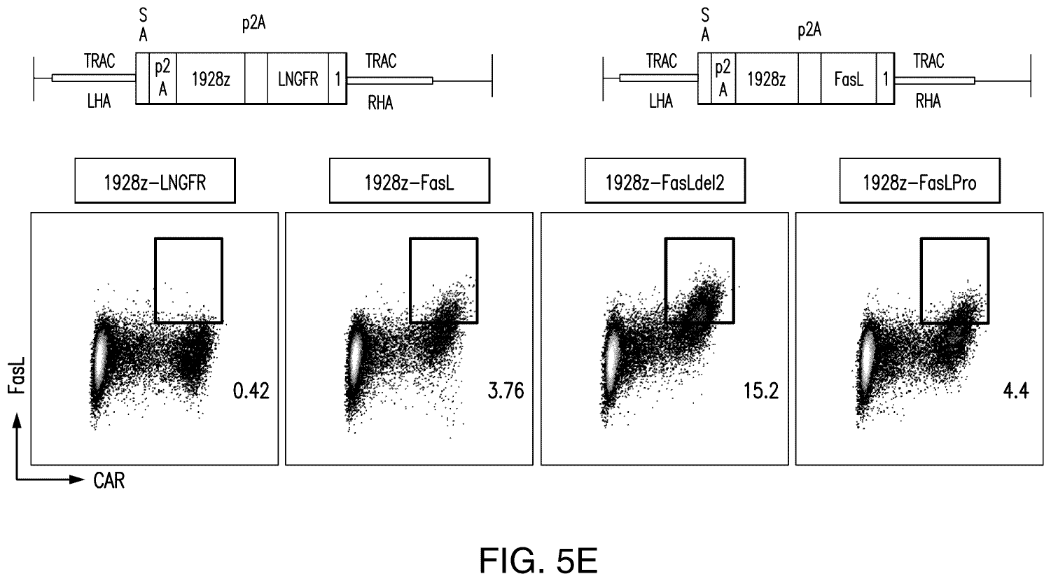

Next, allogeneic CAR T cells capable of inducing active immune tolerance by killing of alloreactive immune effector cells were developed. Active tolerance was obtained through overexpression of Fas ligand (FasL) polypeptides. FIGS. 5A and 5B show a schematic of FasL polypeptides and vectors comprising the same used in the present example. Retroviral constructs overexpressing FasL polypeptides induced increased levels of FasL when detected by fluorescence staining four days after transduction compared to mock transduced cells (black). Surprisingly, FasLdel2 showed the higher expression level.

Next, survival of edited CAR T cells incubated with allogeneic PBMCs was tested. As shown in FIG. 5D, cells with Fas-KO had increased survival in presence of MLR-stimulated allogeneic PBMCs for 24 hours compared to cells edited with TRAC-KO. Surprisingly, transduction with FasL polypeptide, which confers minimal increased surface expression of FasL, did not provide additional benefit. However, transduction of the FasLdel2 variant (SEQ ID NO: 12), which significantly increased FasL expression, led to protection from allogeneic killing.

Finally, AAV bicistronic vectors containing 1928z CAR and FasL were developed. Upon transduction, these vectors led to increased expression of FasL polypeptide in 1928z+cells compared to vector without FasL.

These data show that targeting and knocking out of TRAC and Fas loci along with expression of a protective protein (e.g., a FasL polypeptide) is a successful strategy for the development of allogenic adoptive cell therapies.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunoresponsive cell comprising:
a) an antigen-recognizing receptor that targets an antigen;
b) an exogenous Fas ligand polypeptide (FasL); and
c) a gene disruption of a Fas locus.

2. The immunoresponsive cell of claim 1, wherein the FasL polypeptide is membrane bound.

3. The immunoresponsive cell of claim 1, wherein the FasL polypeptide comprises or consists of an amino acid sequence that is at least about 80% identical to the amino acid sequence set forth in SEQ ID NO: 10, SEQ ID NO: 12, SEQ ID NO: 36, SEQ ID NO: 38, SEQ ID NO: 40, or SEQ ID NO: 42.

4. The immunoresponsive cell of claim 1, wherein the FasL polypeptide comprises or consists of an amino acid sequence set forth in SEQ ID NO: 10, SEQ ID NO: 12, SEQ ID NO: 36, SEQ ID NO: 38, SEQ ID NO: 40, or SEQ ID NO: 42.

5. The immunoresponsive cell of claim 1, wherein (a) the FasL polypeptide comprises a truncated intracellular domain, or (b) the FasL polypeptide does not comprise an intracellular domain.

6. The immunoresponsive cell of claim 1, wherein the FasL polypeptide comprises or consists of an amino acid sequence of amino acids 81 to 281 of SEQ ID NO: 9 or amino acids 81 to 277 of SEQ ID NO: 12.

7. The immunoresponsive cell of claim 1, wherein the antigen-recognizing receptor is a recombinant T cell receptor (TCR), a chimeric antigen receptor (CAR), or a TCR like fusion molecule.

8. The immunoresponsive cell of claim 1, wherein (a) the antigen-recognizing receptor is encoded by a polynucleotide inserted into a first locus within the genome, (b) the FasL polypeptide is encoded by a polynucleotide inserted into a second locus within the genome.

9. The immunoresponsive cell of claim 8, wherein (a) the first locus and/or the second locus are selected from the group consisting of a TRAC locus, a TRBC locus, a TRDC locus, a TRGC locus, and a Fas locus.

10. The immunoresponsive cell of claim 1, wherein the antigen-recognizing receptor and the FasL polypeptide are encoded by a polynucleotide inserted into a first locus within the genome.

11. The immunoresponsive cell of claim 1, wherein the first locus is selected from the group consisting of a TRAC locus, a TRBC locus, a TRDC locus, a TRGC locus, and a Fas locus.

12. The immunoresponsive cell of claim 1, wherein the antigen is a tumor antigen or a pathogen antigen.

13. The immunoresponsive cell of claim 12, wherein the tumor antigen is selected from the group consisting of CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell, epithelial glycoprotein-2 (EGP-2), epithelial glycoprotein-40 (EGP-40), epithelial cell adhesion molecule (EpCAM), receptor tyrosine-protein kinase Erb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, and ERBB.

14. The immunoresponsive cell of claim 1, further comprising a gene disruption of a TRAC locus, a TRBC locus, a TRDC locus, and a TRGC locus, or a combination thereof.

15. The immunoresponsive cell of claim 1, wherein the immunoresponsive cell is a cell of the lymphoid lineage, a cell of the myeloid lineage, or a cell selected from the group consisting of a T cell, a Natural Killer (NK) cell, a B cell, a monocyte, and a macrophage, a pluripotent stem cell from which a lymphoid cell may be differentiated, a pluripotent stem cell from which a myeloid cell may be differentiated, and combinations thereof.

16. The immunoresponsive cell of claim 15, wherein the cell is a T cell or a Natural Killer (NK) cell.

17. The immunoresponsive cell of claim 1, wherein the cell is autologous or allogeneic.

18. A composition comprising the immunoresponsive cell of claim 1.

19. A method for producing a cell of claim 1, the method comprising:
a) generating a gene disruption of a Fas locus in a cell;
b) introducing into the cell a polynucleotide encoding a FasL polypeptide; and
c) introducing into the cell a polynucleotide encoding an antigen-recognizing receptor.

20. A nucleic acid composition comprising a first polynucleotide encoding a Fas ligand polypeptide, and a second polynucleotide encoding a nuclease.

21. The nucleic acid composition of claim 20, wherein
a) the FasL polypeptide comprises or consists of an amino acid sequence set forth in SEQ ID NO: 10, SEQ ID NO: 12, SEQ ID NO: 36, SEQ ID NO: 38, SEQ ID NO: 40, or SEQ ID NO: 42;
b) the FasL polypeptide comprises a truncated intracellular domain; or
c) the FasL polypeptide does not comprise an intracellular domain.

22. A lipid nanoparticle comprising the nucleic acid composition of claim 20.

23. A method of lysing a target cell expressing Fas, comprising contacting the target cell with the immunoresponsive cell of claim 1.

24. A method of reducing tumor burden in a subject, the method comprising administering to the subject an effective amount of the immunoresponsive cells of claim 1.

25. A method of preventing and/or treating a tumor in the subject, administering to the subject an effective amount of the immunoresponsive cells of claim 1.

26. A method of treating a disease or a disorder in a subject, comprising administering to the subject the immunoresponsive cells of any one of claim 1.

27. A kit for reducing tumor burden in a subject, treating and/or preventing a tumor in a subject, and/or increasing or lengthening survival of a subject having a tumor, comprising the cell of claim 1.
